(19) United States
(12) Patent Application Publication
(10) Pub. No.: US 2012/0064179 A1
(43) Pub. Date: Mar. 15, 2012

(54) BICARBONATE SOLUTION FOR BIOAVAILABLE MAGNESIUM AND USES THEREOF

(75) Inventor: Russell Beckett, New South Wales (AU)

(73) Assignee: UNIQUE GLOBAL POSSIBILITIES MEDICAL PTY LIMITED, Sydney, New South Wales (AU)

(21) Appl. No.: 13/266,627

(22) PCT Filed: Apr. 23, 2010
(86) PCT No.: PCT/AU10/00476

§ 371 Date: Nov. 29, 2011

(30) Foreign Application Priority Data
Apr. 30, 2009 (AU) 2009901885

(51) Int. Cl. A61K 33/10; A61P 35/00; A61P 35/04; A61P 9/10; A61P 19/08; A61P 9/00; A61P 9/12; A61P 19/10; A61P 3/10; A61P 11/06; A61P 21/00; A61P 29/00; A61P 31/18; A61P 19/02; A61P 25/06; A61P 25/20; A61P 17/00; A61P 25/00; A61P 3/02

(52) U.S. Cl. 424/686

(57) ABSTRACT

The invention relates to a method of prevention or treatment of a condition such as magnesium deficiency or deficit or abnormality, creatine or creatine phosphate (phosphocreatine) deficiency or abnormality, parathyroid hormone (PTH) excess or abnormality, sodium deficiency or abnormality, potassium deficiency or abnormality, antidiuretic hormone excess or abnormality or a condition characterised by abnormal levels of parathyroid hormone related protein (PTHrP). In the method, a therapeutic quantity of a neutral to mildly alkaline solution of a magnesium salt comprising bicarbonate ions is administered to the patient.

### TECHNICAL FIELD

The present invention relates to bioavailable magnesium, and to methods of using it for the prevention or treatment of pathological conditions.

### BACKGROUND OF THE INVENTION

A typical Western diet is characterised by a chronic, sub-clinical (mild) deficit in magnesium intake. US Department of Agriculture (USDA) statistics on nutrient intakes reveal that 80 per cent of teenage females and 70 per cent of adult females do not meet the recommended daily allowance (RDA) for magnesium intake. About 60 per cent of teenage females and 45 per cent of adult females fall below the 75 per cent RDA mark. The situation is similar in males with 65 per cent of males not meeting the RDA for magnesium intake and 40 per cent of males falling below the 75 per cent RDA mark (see USDA. 1999. Continuing Survey of Food Intakes by Individuals, 1994-1996. Food Surveys Research Group, Agricultural Research Service. http://www.barc.usda.gov/bhnrc/foodsurvey/pdf/Supp.pdf). Numerous epidemiological studies also have evaluated dietary intakes of magnesium and reported that, consistent with USDA findings, magnesium intakes below the RDA are the norm in Western diets.

There have been numerous international prospective studies examining the role of nutritional magnesium deficiency in the diseases of atherosclerosis, ischaemic heart disease, kidney disease and Type 2 diabetes (known collectively as The Metabolic Syndrome). In the USA, these prospective studies include the National Health and Nutrition Examination Survey (NHANES I) involving 8,250 participants; the Atherosclerosis Risk in Communities Study (ARIC) involving 15,792 participants; the Nurses' Health Study (NHS) and Health Professionals' Follow-up Study involving 85,000 female participants and 42,000 male participants; the Women's Health Study (WHS) involving 39,000 female participants; and the Black Women's Health Study involving 59,000 African-American female participants. The conclusions from these studies were that magnesium deficiencies in diet are correlated to atherosclerosis, ischaemic heart disease and Type 2 diabetes (see J. Clin. Epidemiol., Vol. 48 (7), 927-940, 1995; and see American Heart Journal, Vol. 136 (3), 480-490, September, 1998; and see Arch. Intern. Med., Vol. 159, 2151-2159, Oct. 11, 1999; and see Diabetes Care, Vol. 27 (1), 59-65, January, 2004; and see Diabetes Care, Vol. 29 (10), 2238-2243, October, 2006).

A further conclusion from the above studies, and other similar studies, was that dietary levels of magnesium do not correlate to serum levels of magnesium. Correlations between dietary magnesium levels and serum magnesium levels were found to range between r=0.04 and r=0.09 (Pearson correlation coefficient) in all studies (see J. Clin. Epidemiol., Vol. 48 (7), 927-940, 1995; and see American Heart Journal, Vol. 136 (3), 480-490, September, 1998; and see Arch. Intern. Med., Vol. 159, 2151-2159, Oct. 11, 1999). That is, all studies found that there was no correlation. This lack of correlation between standard dietary magnesium intake and serum magnesium levels occurs whether people are either healthy or affected by disease. The lack of correlation is considered to be a result of renal influence on the complexities of magnesium homeostasis when magnesium intake is within the normal dietary range (see J. Clin. Epidemiol., Vol. 48 (7), 927-940, 1995; and see American Heart Journal, Vol. 136 (3), 480-490, September, 1998; and see Arch. Intern. Med., Vol. 159, 2151-2159, Oct. 11, 1999). In diabetes, and other diseases of The Metabolic Syndrome, the lack of correlation between dietary magnesium and serum magnesium, and the low serum magnesium levels evident per se, are considered to be partly the result of increased renal excretion of magnesium due to osmotic diuresis (see Arch. Intern. Med., Vol. 159, 2119-2120, Oct. 11, 1999).

### OBJECT OF THE INVENTION

It is the object of the present invention to at least partially address the above problems.

### SUMMARY OF THE INVENTION

In a first aspect of the invention there is provided a method of prevention or treatment of a condition, said method comprising administering to a patient in need thereof a therapeutic quantity of a neutral to mildly alkaline solution of a magnesium salt, said solution comprising bicarbonate ions. The condition may be magnesium deficiency. It may be a condition that is improved by increasing bioavailable magnesium intake. It may be a deficit in magnesium intake or it may be a deficit in bioavailable magnesium intake. It may be magnesium abnormality. It may be creatine or creatine phosphate (phosphocreatine) deficiency. It may be creatine or creatine phosphate abnormality. It may be parathyroid hormone excess. It may be parathyroid hormone (PTH) abnormality. It may be sodium deficiency. It may be sodium abnormality. It may be potassium deficiency. It may be potassium abnormality. It may be antidiuretic hormone excess. It may be antidiuretic hormone abnormality. It may be a condition characterised by abnormal levels of parathyroid hormone related protein (PTHrP). It may be a combination of any two or more of these conditions.

The following options may be used in conjunction with the first aspect, either individually or in any suitable combination.

The condition may be tissue magnesium deficiency. It may be tissue magnesium abnormality. It may be tissue creatine or tissue creatine phosphate (phosphocreatine) deficiency. It may be tissue creatine or tissue creatine phosphate abnormality. It may be tissue parathyroid hormone excess. It may be tissue parathyroid hormone abnormality. It may be tissue sodium deficiency. It may be tissue sodium abnormality. It may be tissue potassium deficiency. It may be tissue potassium abnormality. It may be tissue antidiuretic hormone excess. It may be tissue antidiuretic hormone abnormality. It may be a combination of any two or more of these conditions.

The condition may be one that is improved by increasing magnesium intake or it may be one that is improved by increasing bioavailable magnesium intake.

The condition may be one that is improved by increasing body anabolism or it may be one that is improved by decreasing body catabolism or it may be one that is improved by both increasing body anabolism and by decreasing body catabolism. The condition may be one that is improved by increasing cell anabolism or it may be one that is improved by decreasing cell catabolism or it may be one that is improved by both increasing cell anabolism and by decreasing cell catabolism.

The condition may be selected from the group consisting of ethanol toxicity, methyl xanthine toxicity, migraine, sleeplessness, psychiatric conditions requiring a calmative, pancreatitis and skin conditions. It may be traumatic injury to a muscle, bone and/or joint due to, for example, car accident, other accident, sports injury etc. It may be chronic headaches. It may be a mood disorder. It may be an aberration in acid base balance (e.g. as indicated by urinary pH, particularly low urinary pH). The condition may be arterial intima media thickness (IMT) particularly carotid arterial intima media thickness (cIMT). It may be a combination of any two or more of these conditions.

The condition may be any other condition that is improved by increasing bioavailable magnesium intake.

The pH of the solution may be between about 7.0 and about 9.5. It may be between about 8.3 and about 8.5.

The magnesium concentration may be between about 50 and about 250 mg/L magnesium ions.

The solution may comprise about 120 mg/L magnesium ions.

The solution may comprise about 200 to about 1500 mg/L bicarbonate ions.

The method may additionally comprise reducing or ceasing activity which causes said condition. The method may additionally comprise reducing or ceasing activity which exacerbates or aggravates said condition.

The administering may comprise orally administering.

The administering may be at the rate of about 1 to about 2 litres per day.

The administering may be in sufficient quantity to provide between about 50 to about 300 mg magnesium per day to said patient.

The administration may be continued for sufficient time for the condition to be alleviated. It may be continued indefinitely.

The patient may be suffering a second condition, said second condition being one for which an increase in parathyroid hormone is adverse, and where said method does not result in a significant increase in parathyroid hormone. The second condition may be selected from the group consisting of high blood pressure, ischemic heart disease, osteoporosis, atherosclerosis, The Metabolic Syndrome, Type 2 diabetes, asthma, osteoarthritis and wear and tear injuries to muscles, bones and joints. In the event that patient is suffering a second condition, the first condition may be an inflammatory disease or a degenerative disease. It may be traumatic injury to a muscle, bone and/or joint due to, for example, car accident, other accident, sports injury etc. The condition may be arterial intima media thickness (IMT) particularly carotid arterial intima media thickness (cIMT). It may be a combination of any two or more of these conditions.

The condition may be any other condition that is improved by increasing bioavailable magnesium intake.

The first condition may for example be senescence or it may be arthritis.

In a second aspect of the invention there is provided the use of a neutral to mildly alkaline solution of a magnesium salt, said solution comprising bicarbonate ions, for the prevention or treatment of a condition.

The condition may be magnesium deficiency. It may be a deficit in magnesium intake or it may be a deficit in bioavailable magnesium intake. It may be magnesium abnormality. It may be creatine or creatine phosphate (phosphocreatine) deficiency. It may be creatine or creatine phosphate abnormality. It may be parathyroid hormone excess. It may be parathyroid hormone abnormality. It may be sodium deficiency. It may be sodium abnormality. It may be potassium deficiency. It may be potassium abnormality. It may be antidiuretic hormone excess. It may be antidiuretic hormone abnormality. It may be a condition characterised by abnormal levels of parathyroid hormone related protein (PTHrP). It may be a combination of any two or more of these conditions.

A patient to whom the solution is administered may additionally suffer a second condition, said second condition being one for which an increase in parathyroid hormone is adverse. The second condition may be selected from the group consisting of high blood pressure, ischemic heart disease, osteoporosis, atherosclerosis, The Metabolic Syndrome, Type 2 diabetes, asthma, osteoarthritis and wear and tear injuries to muscles, bones and joints.

In a third aspect of the invention there is provided a method for modulating the response of the parathyroid gland of a patient to a change in extracellular calcium concentration, said method comprising administering to said patient a therapeutic quantity of a neutral to mildly alkaline solution of a magnesium salt, said solution comprising bicarbonate ions.

The modulating may comprise reducing the response.

In a fourth aspect of the invention there is provided the use of a neutral to mildly alkaline solution of a magnesium salt, said solution comprising bicarbonate ions, for modulating the response of the parathyroid gland of a patient to a change in extracellular calcium concentration.

In a fifth aspect of the invention there is provided a method of prevention or treatment of a condition, said method comprising administering to a patient in need thereof a therapeutic quantity of a neutral to mildly alkaline solution of a magnesium salt, said solution comprising bicarbonate ions, said condition being selected from the group consisting of atherosclerosis, vascular calcification, osteoarthritis and cancer.

The cancer may be breast cancer or prostate cancer, or may be some other cancer.

In a sixth aspect of the invention there is provided a method for preventing or inhibiting metastasis to bone of a cancer, said method comprising administering to a patient in need thereof a therapeutic quantity of a neutral to mildly alkaline solution of a magnesium salt, said solution comprising bicarbonate ions.

The cancer may be breast cancer or prostate cancer, or may be some other cancer.

In a seventh aspect of the invention there is provided the use of a neutral to mildly alkaline solution of a magnesium salt, said solution comprising bicarbonate ions, for the prevention or treatment of a condition selected from the group consisting of atherosclerosis, vascular calcification, osteoarthritis and cancer. The cancer may be breast cancer or prostate cancer, or may be some other cancer.

In an eighth aspect of the invention there is provided the use of a neutral to mildly alkaline solution of a magnesium salt, said solution comprising bicarbonate ions, for preventing or inhibiting metastasis to bone of a cancer. The cancer may be breast cancer or prostate cancer, or may be some other cancer.

In the methods of the invention outlined above, the therapeutic quantity may be at or near the recommended daily allowance (RDA) for magnesium. The RDA (or RDI—recommended daily intake) may be that RDA applicable in the country in which the solution is administered. Representative RDAs for magnesium include 400 mg (USA) and 320-420 mg (Australia and New Zealand, depending on particulars of the subject—age, sex etc.)

In the methods, it is preferred that no pathology resulting from said method be identified in said patient.

In a ninth aspect of the invention there is provided use of a magnesium salt and a source of carbonate or bicarbonate ions for the manufacture of a medicament for the treatment of a condition which is improved by increasing bioavailable magnesium intake. The condition may be magnesium deficiency. It may be a deficit in magnesium intake. It may be a deficit in bioavailable magnesium intake. It may be magnesium abnormality. Representative conditions are described elsewhere in this specification.

In a tenth aspect of the invention there is provided a method of improving acid/base balance and/or arterial intima media thickness (IMT), said method comprising administering to a patient in need thereof a therapeutic quantity of a neutral to mildly alkaline solution of a magnesium salt, said solution comprising bicarbonate ions.

### BRIEF DESCRIPTION OF THE DRAWINGS

Preferred embodiments of the present invention will now be described, by way of example only, with reference to the accompanying drawings wherein:

FIG. 1 is a graph showing serum magnesium over time;

FIG. 2 is a graph showing serum parathyroid hormone over time;

FIGS. 3 and 4 are graphs showing serum potassium over time;

FIG. 5 is a graph showing serum sodium over time;

FIG. 6 is a graph showing serum albumin over time; and

FIG. 7 is a graph showing urinary pH over time.

### DETAILED DESCRIPTION OF THE INVENTION

The invention provides a method of prevention or treatment of a condition, said method comprising administering to a patient in need thereof a therapeutic quantity of a neutral to mildly alkaline solution of a magnesium salt. The magnesium solution additionally comprises bicarbonate ions. Thus the solution may be a magnesium bicarbonate solution. The treatment is suitable for preventing or treating the following conditions: magnesium deficiency, deficit in magnesium intake or deficit in bioavailable magnesium intake, magnesium abnormality, creatine or creatine phosphate (phosphocreatine) deficiency, creatine or creatine phosphate abnormality, parathyroid hormone excess, parathyroid hormone abnormality, sodium deficiency, sodium abnormality, potassium deficiency, potassium abnormality, antidiuretic hormone excess and antidiuretic hormone abnormality. The treatment may be suitable for treating patients that exhibit more than one of the above conditions. The abnormality or deficiency or excess may be a tissue abnormality or tissue deficiency or excess, or it may be a plasma abnormality or plasma deficiency or excess. These conditions may be related to (may be, or may result from, or may cause) ethanol toxicity, methyl xanthine toxicity, migraine, sleeplessness, psychiatric conditions requiring a calmative, pancreatitis or a skin condition, or traumatic injury to a muscle and/or bone and/or joint due to, for example, car accident, other accident, sports injury etc. or chronic headaches or a mood disorder. The condition may be arterial intima media thickness (IMT) particularly carotid arterial intima media thickness (cIMT). It may be a combination of any two or more of these conditions.

The condition may be any other condition that is improved by increasing bioavailable magnesium intake.

In the present specification, where reference is made to “prevention or treatment” (or “preventing or treating” or related phrases) this may refer separately to preventing the condition or to treating the condition, or it may refer to a single treatment that may prevent or treat the condition (or both prevent and treat the condition) as appropriate under the prevailing conditions.

The inventor has found that the above conditions may be improved by increasing body anabolism or by decreasing body catabolism or by both. The inventor has found that the above conditions may be improved by increasing cell anabolism or by decreasing cell catabolism or by both. This may be achieved by administering a source of bioavailable magnesium. This source may be the solution of magnesium described herein.

The pH of the solution may be between about 7.0 and about 9.5, or about 7.0 to 9.0, 7.0 to 8.5, 7.0 to 8.0, 7.0 to 7.5, 7.5 to 9.5, 8.0 to 9.5, 8.5 to 9.5, 9.0 to 9.5, 7.5 to 9.0, 7.5 to 8.5, 7.5 to 8.0, 8.0 to 9.0, 8.5 to 9.0, 8.0 to 8.5 or 8.3 to 8.5, e.g. about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4 or 9.5. The magnesium concentration may be between about 50 and about 250 mg/L magnesium ions, or about 50 to 200, 50 to 150, 50 to 100, 100 to 250, 150 to 200, 200 to 250, 100 to 200, 100 to 150 or 150 to 200, e.g. about 50, 60, 70, 80, 90, 100, 110, 120, 130, 140, 150, 160, 170, 180, 190, 200, 210, 220, 230, 240 or 250 mg/L. The solution may comprise about 200 to about 1500 mg/L bicarbonate ions, or about 500 to 1500, 1000 to 1500, 200 to 1000, 200 to 500 or 500 to 1000 mg/L, e.g. about 200, 300, 400, 500, 600, 700, 800, 900, 1000, 1100, 1200, 1300, 1400 or 1500 mg/L. Suitable solutions and methods for making them are described in WO98/41218 and WO03/086973, the contents of which are incorporated herein by cross-reference. The solution may have other ions than magnesium and bicarbonate, e.g. sodium, potassium, chloride etc. Alternatively there may be no significant quantity, or no substantial quantity, of ions in the solution other than magnesium and bicarbonate, and optionally also carbonate. These ions may be in non-harmful concentrations. The solution itself may be non-harmful. Each of the solutes in the solution may be in a non-harmful concentration. In this context a “non-harmful” concentration may be a non-toxic concentration. It may be a concentration at which no harmful effects are caused when the solution is administered to the patient under a dosage regime appropriate for the condition to be treated. In some instances some minor harmful effects may be caused by administration of the solution, in which case the term “non-harmful” may be taken to refer to a concentration at which any harmful effects are outweighed by a benefit provided by administration of the solution to the patient.

In some embodiments the solution used in the invention consists, or consists essentially, of water, magnesium ions and bicarbonate ions. In other embodiments the solution consists, or consists essentially, of water, magnesium ions, bicarbonate ions and possibly carbonate ions and/or carbon dioxide. In yet other embodiments the solution contains no components, or essentially no components, other than water, magnesium ions, bicarbonate ions, carbonate ions, carbon dioxide and dissolved gases from the air. In this context the term “consists essentially of” indicates that any other materials present are not intentionally added and are in sufficiently low concentration as to have no effect on the operation of the invention. Similarly the term “essentially no components other than” indicates that any other components are not intentionally added and are in sufficiently low concentration as to have no effect on the operation of the invention.

The solution used in the present invention may be made by a process comprising (or consisting of) dissolving the required amount of magnesium carbonate in water using the minimum amount of carbon dioxide gas required to achieve dissolution of the carbonate and production of the bicarbonate and to achieve the desired pH (described elsewhere herein). The carbon dioxide may be passed through (e.g. bubbled through) a mixture of the magnesium carbonate and water. It may be passed therethrough for sufficient time to achieve the desired pH and achieve dissolution. The mixture of water and magnesium carbonate may be agitated (e.g. stirred, swirled, shaken, sonicated, ultrasonicated etc.) during the process of dissolution. Alternatively the mixture may not be agitated. The passing of the carbon dioxide through the mixture may provide agitation sufficient to achieve dissolution without the need for other agitation. Thus no agitation other than that provided by the passing of the carbon dioxide through the mixture may be applied. This process may be conducted at a temperature of about 0 to about 25° C., or about 0 to 20, 0 to 10, 0 to 5, 5 to 25, 10 to 25, 15 to 25 or 15 to 20° C., or about 0, 1, 2, 3, 4, 5, 6, 7, 8, 9, 10, 11, 12, 13, 14, 15, 16, 17, 18, 19, 20, 21, 22, 23, 24 or 25° C.

The method of treatment may additionally comprise reducing or ceasing activity which causes said condition. Thus for example if the condition is alcohol toxicity, the patient should preferably refrain from consuming alcohol until the treatment is completed, or until symptoms of alcohol toxicity (e.g. headache, dehydration) subside or disappear. Similarly, if the condition is methyl xanthine toxicity, the patient should preferably refrain from consuming foods containing methyl xanthines (e.g. chocolate or tea or coffee or cola or caffeine) until the symptoms subside or disappear.

Commonly the solution of the present invention is administered orally. Due to the volume of daily administration, it is often inconvenient to administer via other routes, however such other routes are contemplated by the present invention. The most common form of administration therefore is for the patient to drink the solution. Thus the administration may be self-administration by the patient.

A suitable administration rate of the solution of the invention is about 1 to about 2 litres per day, or may be about 1 to 1.5, 1.5 to 2 or 1.3 to 1.7 litres per day, e.g. about 1, 1.1, 1.2, 1.3, 1.4, 1.5, 1.6, 1.7, 1.8, 1.9 or 2 litres per day. The rate of administration may be sufficient to provide between about 50 and about 300 mg magnesium per day to the patient, or about 50 to 250 or 50 to 200 or 50 to 100 or 100 to 300 or 100 to 250 or 100 to 200 or 150 to 250 or 180 to 220 or 200 to 300 mg per day. The treatment is preferably continued for sufficient time for the condition to be alleviated. This may depend on the nature of the condition. It may be for example for about 1 day, or may be for about 2, 3, 4, 5, 6 or 7 days, or for more than 7 days. In some cases it is necessary to continue treatment for long periods, e. g. possibly one or several months (e.g. about 1, 2, 3, 4, 5 or 6 months) or possibly indefinitely, in order to prevent recurrence of the condition or to reduce the likelihood of such recurrence.

The present invention is well suited to cases in which the patient is suffering a second condition, said second condition being one for which an increase in parathyroid hormone is adverse. This is because other measures that may be capable of raising magnesium levels (such as administration of a comparable quantity of a placebo comprising water that does not contain magnesium and bicarbonate ions in the quantities present in the solution of this invention) also result in an increase in parathyroid hormone. By contrast, the solution used in the present method stabilises parathyroid hormone and does not result in a significant increase therein. Thus conditions that are caused or exacerbated by an increase in parathyroid hormone are not contraindicated, and may in some cases be treated or prevented, by the present method. Conditions for which an increase in parathyroid hormone may be adverse include high blood pressure, ischemic heart disease, osteoporosis, atherosclerosis The Metabolic Syndrome, Type 2 diabetes, asthma, osteoarthritis and wear and tear injuries to muscles, bones and joints. Patients suffering from any one or more of these may be safely treated with the present method without the likelihood that the treatment exacerbates that condition (or those conditions).

Thus the inventor has found that various specific magnesium bicarbonate solutions, as described herein, are sources of bioavailable magnesium. Consequently the consumption of such solutions increases plasma (serum) magnesium concentrations. Consumption of these solutions has also been found to increase plasma (serum) creatinine concentrations and increase creatine and creatine phosphate (phosphocreatine) concentrations in body cells. These solutions have also been found to stabilise plasma (serum) parathyroid hormone concentrations in the body. They may also decrease urine inorganic phosphate concentrations in the body. Consumption of the solutions also increases plasma (serum) sodium concentrations and increases plasma (serum) potassium concentrations in the body. Thus these solutions can increase plasma (serum) potassium concentrations which increases interstitial (extracellular) potassium concentrations, maintains cell volume and protects body cells from apoptosis and decreases the magnitude of cell resting membrane potentials. The solutions can also blunt the secretion of antidiuretic hormone (ADH, vasopressin) in the body.

Thus the solutions described herein can be utilised to prevent and to treat disease conditions and to prevent and to treat pathological conditions in humans and other animals where the disease conditions and the pathological conditions involve either tissue magnesium deficiency or abnormality, or tissue creatine or creatine phosphate (phosphocreatine) deficiency or abnormality, or tissue parathyroid hormone excess or abnormality, or tissue sodium deficiency or abnormality, or tissue potassium deficiency or abnormality, or tissue antidiuretic hormone (ADH, vasopressin) excess or abnormality. They may also be used to increase anabolic processes in the body and decrease catabolic processes in the body.

The magnesium bicarbonate solutions of the present invention are neutral to mildly alkaline. They may have pH values between pH 7.0 and pH 9.5. Magnesium cations may be present in concentrations from 50 mg per liter to 250 mg per liter of solution. Bicarbonate anions may be present in concentrations from 200 mg per liter to 1,500 mg per liter of solution.

WO98/41218 describes solutions of magnesium salts which are neutral to mildly basic. Subsequently, WO03/086973 described methods for producing those solutions. The present specification describes the used of these solutions for the treatment of pathological conditions in a patient. The patient may be a human. The patient may be a non-human. The patient may be a non-human mammal, e.g. a dog, a cat, a horse, a cow, a sheep, a pig or some other mammal. The patient may be a domesticated animal. The patient may be a wild or non-domesticated animal. The patient may be a farm animal.

### Elevated Parathyroid Hormone Concentrations, Alterations in Calcium Homeostasis and Disease

Extracellular calcium concentrations in humans and other mammals are tightly regulated within a narrow physiological range in order to provide for the proper functioning of heart and skeletal muscles, the proper functioning of the brain, nerves and synapses, the proper functioning of hormone secretion, and the proper functioning of platelet aggregation and blood coagulation. Intracellular calcium concentrations in humans and other mammals are even more exquisitely regulated in order for calcium to serve as an intracellular second messenger in the regulation of cell division, muscle cell contraction, cell motility, cell membrane trafficking, cell secretion and exocytosis (see Basic & Clinical Endocrinology, Greenspan F. S. and Gardner D. G., pg 295, Seventh Edition, 2004, McGraw-Hill, New York).

The extracellular to intracellular concentration gradient of calcium across cell membranes is ten thousand to one (10,000 to 1). Ionised plasma calcium concentration, and calcium concentration outside body cells, is about 1.2 mmol/litre. Intracellular free calcium concentration is 0.00018 mmol/litre (180 nmol/litre). In medicine, the ubiquitous and universal indicator of cell death is the point where a cell can no longer sustain and maintain the large calcium concentration gradient across the cell membrane. At this point, cells become calcified. It is not known whether cell death precedes an influx of calcium or whether an influx of calcium is the precursor to, or signal for, cell death (see Pathology, Rubin, E. and Farber, J. L., editors, pg 17, Third Edition, 1999, Lippincott-Raven Publishers, Philadelphia). In any case, cell death and cell calcification lead to organ pathology and disease.

Parathyroid hormone regulates extracellular calcium concentrations. Decreased extracellular calcium concentrations in the body lead to an increase in the secretion of parathyroid hormone. Increased parathyroid hormone concentrations stimulate the release of calcium and phosphate from bone and activate retention of calcium by the kidney. In relation to bone, increased parathyroid hormone concentrations maintain blood and extracellular calcium concentrations by activating new bone remodeling units, though as a result of the inherent inefficiency of this process increased bone remodeling leads to accelerated bone loss and osteoporosis (see Basic & Clinical Endocrinology, Greenspan F. S. and Gardner D. G., pgs 335-338, Seventh Edition, 2004, McGraw-Hill, New York). Continuous elevated concentrations of parathyroid hormone are detrimental to the body.

Progressive deficits in renal and intestinal function impair calcium homeostasis during normal human aging. These deficits include progressive inefficiency of vitamin D production by the skin and the progressive inefficiency of the conversion of vitamin D to its active form (1,25-dihydroxyvitamin D) in the kidney. Consequently, intestinal calcium absorption becomes progressively less efficient leading to modest reductions in plasma ionised calcium with compensatory hypersecretion of parathyroid hormone (see Basic & Clinical Endocrinology, Greenspan F. S. and Gardner D. G., pg 338, Seventh Edition, 2004, McGraw-Hill, New York). Parathyroid hormone maintains blood calcium concentrations by stimulating the release of calcium and phosphate from bone.

Many diseases in the body have metabolic components as major parameters in disease pathogenesis. These diseases include the diseases of The Metabolic Syndrome such as atherosclerosis, ischemic heart disease, kidney disease and Type 2 diabetes. In addition, it is now recognised that both the diseases osteoporosis and osteoarthritis have metabolic components in their pathogenesis. Indeed, osteoporosis is one of the most common metabolic diseases per se and is the most common medical problem in older women occurring in fifty per cent of women in their lifetime.

In osteoarthritis, there is metabolic activity associated with increased thickness of the subchondral bone. There are large peripheral growths of bone and cartilage called osteophytes which are thought to represent the bone's attempt to grow a new articular surface. Although cartilage pathology has been traditionally thought to be the primary cause of osteoarthritis, it is now considered that the metabolism and structure of bone adjacent to a joint is important in the proper maintenance of articular cartilage. Aberrations in bone metabolism adjacent to joints contribute significantly to osteoarthritis (see Rheumatology (Oxford), Vol. 42(11), 1311-1316, November, 2003).

In general, those diseases with metabolic components in disease pathogenesis such as The Metabolic Syndrome, osteoporosis and osteoarthritis have pathologies that can be attributed to either increases in parathyroid hormone concentrations or to aberrations in calcium homeostasis or to aberrations in calcium and phosphate homeostasis or turnover or to the dystrophic deposition of calcium.

There are two cell receptors for parathyroid hormone. The first receptor that was identified recognises, and is activated by, both parathyroid hormone and parathyroid hormone related protein (PTHrP) and is designated the PTH-1 receptor. The PTH-2 receptor is activated by parathyroid hormone only. The PTH-1 receptor is widely expressed by the cells of many tissues and appears to be involved in tissue pathology when either overstimulated by or bound to parathyroid hormone related protein, parathyroid hormone or parathyroid hormone fragments.

The PTH-1 receptor in kidney and bone is an 80,000 MW glycoprotein member of the G protein receptor superfamily. In bone, the PTH-1 receptor is absent on osteoclasts (the bone cells responsible for bone resorption and calcium and phosphate release). This means that other bone cells that contain PTH-1 receptors, such as osteoblasts (the bone forming cells), pass the parathyroid hormone signal to the osteoclasts for bone resorption and calcium and phosphate release. This complex interaction allows for bone formation to be activated along with bone resorption. However, small bone deficits persist on completion of each cycle of bone resorption (see Basic & Clinical Endocrinology, Greenspan F. S. and Gardner D. G., pg 334, Seventh Edition, 2004, McGraw-Hill, New York). Eventually, macroscopic bone loss and osteoporosis result.

It is known that the PTH-1 receptor is expressed in various tissues including blood vessels and cartilage. The PTH-1 receptor on chondrocytes is considered to play a role in regulating proliferation and differentiation of chondrocytes in the osteophytes of arthritis (see Histochem. Cell. Biol., Vol. 119 (4), 281-287, April, 2003). It is known that parathyroid hormone related protein (PTHrP) is up-regulated in osteoarthritis (see Osteoarthritis Cartilage, Vol. 13 (5), 395-404, May, 2005). The PTH-1 receptor on stem cells and smooth muscle cells in the walls of blood vessels is considered to play a role in atherosclerosis and vascular calcification (see Circ. Res. Vol. 99 (10), 1044-1059, Nov. 10, 2006; and see Stroke, Vol. 34 (7); 1783-1789 July, 2003; and see Atherosclerosis, Vol. 198 (2), 264-271, June, 2008; and see Am. J. Physiol. Renal Physiol., Vol. 292 (4), 1215-1218, April, 2007). It is not known whether parathyroid hormone per se or fragments of parathyroid hormone play a role in calcification of arteries, though some studies have demonstrated that parathyroid hormone directly stimulates endothelial expression of atherosclerotic parameters which lead to atherosclerosis (see Am. J. Physiol. Renal Physiol., Vol. 292 (4), 1215-1218, April, 2007).

In atherosclerosis, calcifications occur in the intima and media of blood vessels supplying the heart, brain, kidneys, lower extremities and small intestine. Myocardial infarction (heart attack), cerebral infarction (stroke), and aortic aneurysms are the major consequences of this disease. Atherosclerosis, through calcification of arteries and diminished arterial perfusion, causes ischemic heart disease, sudden cardiac death, gangrene of the legs, mesenteric occlusion, and cerebrovascular disease (ischemic encephalopathy).

Constant elevated levels of parathyroid hormone or its fragments may either overstimulate or bind to PTH-1 receptors in bone, joints, blood vessels and other tissues. This contributes to tissue pathology and disease including osteoporosis, osteoarthritis, atherosclerosis, diseases correlated to senescence and aberrant tissue calcifications and calcium phosphate deposits.

### Elevated Antidiuretic Hormone (ADH, Vasopressin) and Disease

Regulation of water balance in the body involves an interaction between osmotic and volume stimuli. Osmoreceptors in the hypothalamus detect a rise in plasma osmolality and trigger the release of antidiuretic hormone (ADH, vasopressin). Antidiuretic hormone is synthesised and secreted by the hypothalamo-neurohypophysial system. An increase in antidiuretic hormone leads to the absorption of water from the collecting duct system of the kidneys. When antidiuretic hormone is present, epithelial permeability increases markedly and water is reabsorbed. The absorption of water from the kidneys assists in the prevention of body dehydration. Antidiuretic hormone exercises its effects on the collecting duct system of the kidneys by stimulating vasopressin V₂ receptors.

The stimulation of V₂ receptors by antidiuretic hormone (vasopressin) leads to the absorption of water from the collecting duct system of the kidneys. The stimulation of V₁ receptors by vasopressin leads to vasoconstriction, glycogenolysis, platelet aggregation, ACTH release, prostaglandin synthesis and growth of vascular smooth muscle cells. Activation of vasopressin V₁ receptors increases phosphatidylinositol breakdown which causes cellular calcium mobilisation (see Basic & Clinical Endocrinology, Greenspan F. S. and Gardner D. C., pg 163, Seventh Edition, 2004, McGraw-Hill, New York). Vasopressin stimulates vascular smooth muscle cell growth by increasing the expression of the proto-oncogenes c-fos and c-jun. Few mutations of c-jun have been described in human tumors, but over-expression of the gene protein has been described in lung and colorectal cancers (see Pathology, pg 183, Third Edition, 1999, Lippincott-Raven, Philadelphia). The expression of c-fos occurs in teratocarcinomas.

Vasopressin is involved in the cellular proliferation of breast, pulmonary and pancreatic cancers (see Urologic Oncology: Seminars and Original Investigations, 2009—in press). These cancers express V₂ receptors on the cell surface. Vasopressin gene expression in cancer cells leads to production of both normal and abnormal forms of tumour vasopressin mRNA and proteins (see Experimental Physiology, Vol. 85 (Suppl 1), 27S-40S, 2000). It has been found in vitro that vasopressin has a proliferative effect by acting on the V₂ receptor and that selective vasopressin antagonists blocking the V₂ receptor prevent cancer cell growth (see Urologic Oncology: Seminars and Original Investigations, 2009—in press; and see Br. J. Pharmacol., Vol. 156 (1), 36-47, January, 2009).

Vasopressin is a potent coronary vasoconstrictor and vasopressin-induced myocardial ischemia often occurs (see Goodman & Gilman's The Pharmacological Basis of Therapeutics, pgs 721-725, Ninth Edition, International Edition, 1996, McGraw-Hill, New York). Over stimulation of vasopressin V₁ and V₂ receptors is correlated to several diseases including atherosclerosis and ischemic heart disease. The blunting of the secretion of vasopressin decreases the effects of vasopressin on both V₁ and V₂ receptors.

It has been shown that alcohol (ethanol) intoxication, and associated headache and hangover, are associated with metabolic acidosis and a rapid loss of magnesium from the blood and brain (see Alcohol, Vol. 12 (2), 131-136, March-April, 1995; and see Alcohol, Vol. 19 (2), 119-130, October, 1999). Ethanol inhibits antidiuretic hormone (ADH, vasopressin) during initial alcohol intoxication which leads to diuresis, loss of electrolytes and dehydration. Later, the severity of the hangover resulting from alcohol intoxication is correlated to increased concentrations of antidiuretic hormone (see Ann. Intern. Med., Vol. 132 (11), 897-902, Jun. 6, 2000).

### Apoptosis, Decreased Extracellular Potassium and Disease

Apoptosis is associated with the major diseases of atherosclerosis, ischaemic heart disease, Type 2 diabetes, osteoporosis and osteoarthritis. Apoptosis is associated with metabolic catabolism and degenerative processes such as senescence and the aging process. Excessive apoptosis is associated with degenerative diseases, ischemic injury such as myocardial infarction and stroke, and virus-induced lymphocyte depletion such as human immunodeficiency virus (HIV) infection and Acquired Immunodeficiency Syndrome (AIDS) (see Robbins Pathologic Basis of Disease, pgs 18-25, Sixth Edition, 1999, W.B. Saunders Company, Philadelphia).

Progressive deficits in renal function impair calcium, sodium and potassium homeostasis during normal human aging. The functional unit of the kidney is the kidney nephron. With aging, kidney nephron losses occur and individual nephron function diminishes. With aging, and particularly with medications consumed by the aged and aging, plasma and extracellular potassium concentrations may be decreased considerably. Decreased extracellular potassium concentrations are correlated to decreases in cell hydration, decreases in cell volume and activation of apoptotic processes (see Arch. Biochem. Biophys., Vol. 462 (2), 176-188, Jun. 15, 2007; and see Lancet, Vol. 341 (8856), 1330-1332, May 22, 1993). Excessive activation of apoptotic processes leads to cell loss in all organs, including vital organs such as the brain. Diseases correlated to tissue and organ degeneration and diseases correlated to aging and senescence become manifest.

Potassium channels in cell membranes constitute a large and heterogeneous family of proteins that control cell plasma membrane potential. A small elevation in extracellular potassium concentration decreases the K⁺ᵢₙ/K⁺ₒᵤₜ ratio and partially depolarises the cell plasma membrane—that is, makes the resting potential less electronegative. Potassium channels are recognised as potential therapeutic targets in the treatment of a range of brain and psychiatric diseases, heart diseases, muscle, bone and joint diseases, diseases associated with tumorigenesis and diseases of The Metabolic Syndrome, including diabetes (see Recent Patents CNS Drug Discov., 2(3):200-28, November 2007).

### EXAMPLE 1

A double-blind, placebo-controlled clinical trial involving 70 patients with a range of medical conditions was conducted. Changes were evaluated in biochemical, physiological and pathological biomarkers that had resulted from the consumption of various specific aqueous alkaline magnesium bicarbonate solutions.

Participants in the trial were permitted to continue regular medications for their medical conditions including hormone replacement therapy, proton pump inhibitors, H2-blockers, glucosamine, analgesics and anti-cholesterolaemia medication provided the dose remained stable. Significant physical or mental illness precluded participation in the trial.

All magnesium bicarbonate solutions were consumed in amounts that resulted in daily magnesium intakes within the recommended daily allowance (RDA) for magnesium. The clinical trial was conducted over 18 months and each patient consumed either magnesium bicarbonate solution or placebo for 12 weeks. The magnesium bicarbonate was consumed in spring water (devoid of sodium and potassium) in amounts that resulted in the consumption of between 50 mg and 300 mg magnesium per day. The placebo consisted of spring water only (devoid of magnesium and bicarbonate and devoid of sodium and potassium) and was consumed in amounts of 1 to 2 litres per day. Each patient in the trial had samples taken for biomarker assessment over 4 visits.

The clinical trial was conducted at St Vincent's Hospital, Sydney, Australia, under the auspices of The University of New South Wales and under registration from the Therapeutic Goods Administration (TGA) of the Australian Government Department of Health and Ageing. TGA Clinical Trial Number: 2005/448. International Clinical Trial Registration ACTRN 12609000863235. The Drug Safety and Evaluation Branch of the TGA acknowledged the clinical trial before patients commenced the study treatments. The clinical trial was conducted in accordance with ICH Harmonized Tripartite Guidelines for Good Clinical Practice 1996 (CPMP/ICH/135/95); US Code of Federal Regulations dealing with clinical studies (21 CFR including parts 50 and 56); and Declaration of Helsinki (V11 October, 2000).

The various specific magnesium bicarbonate solutions consumed in the clinical trial were aqueous alkaline solutions with pH values between pH 7.0 and pH 9.5 and magnesium cations present in concentrations from 50 mg per liter to 250 mg per liter of solution and bicarbonate anions present in concentrations from 200 mg per liter to 1,500 mg per liter of solution.

### Statistics

The statistics for the clinical trial incorporated a general statistical methodology, a supportive repeated measures analyses of variance and additional analyses of change as required.

Biochemical biomarkers, physiological biomarkers and pathological biomarkers were taken as continuous variables. The continuous variables were summarised and tabulated at each of the 4 visits giving the number of observations, mean, standard deviation, minimum, median and maximum values of the variables for both the magnesium bicarbonate group and the placebo group. The change from baseline to Day 84 was compared between groups for all biomarkers. Measurements were compared using independent two sample t-tests or their non-parametric equivalent. Supportive repeated measures analyses of variance were performed. Additional analyses of change in biomarkers were performed to examine i) the magnitude of the absolute differences in means between groups ii) the magnitude of the differences between groups in the trends of means iii) the magnitude of the trend of means in all participants independent of group and iv) the correlation between changes in each variable with changes in the other variables.

### Safety

It is stated by the relevant medical officers in the magnesium bicarbonate Clinical Trial Report that overall magnesium bicarbonate consumption was well tolerated by the subjects who volunteered for the clinical trial. The consumption of magnesium bicarbonate passed all clinical, biochemical, urine and blood testing safety criteria.

A range of safety biomarkers were assessed in the clinical trial. Approximately 40 clinical, biochemical, urine and blood safety criteria were completed. There was no statistically significant pathology identified with any of the safety biomarkers for either the group consuming magnesium bicarbonate or the group consuming placebo.

### Magnesium

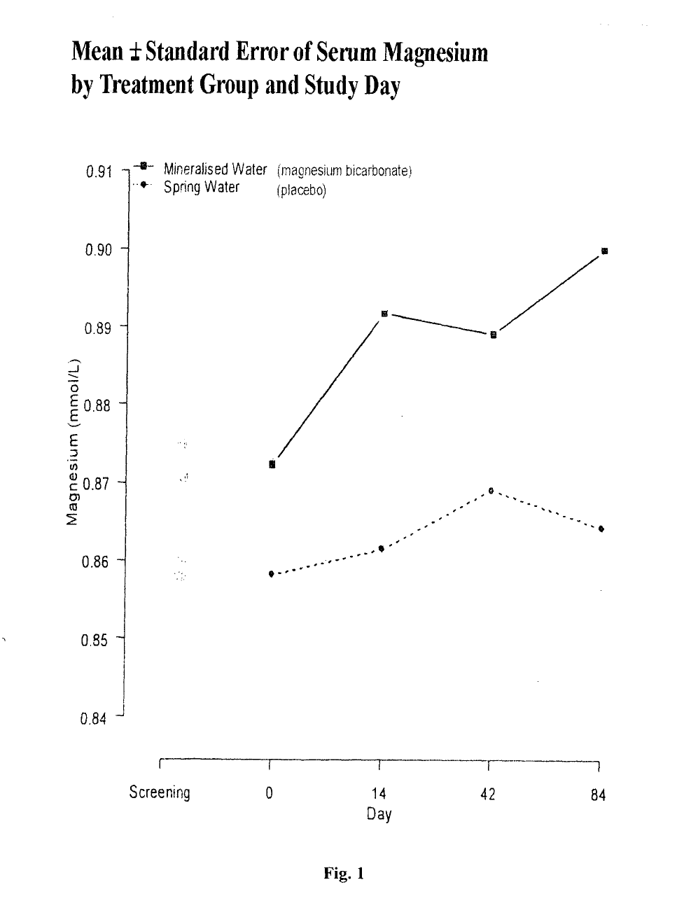

In the clinical trial, there was a statistically significant difference in change from baseline to Day 84 in the mean level of serum magnesium (p=0.027) between the two treatment groups. This is shown graphically in FIG. 1. The mean increase in serum magnesium was 0.022 mmol/L (95% CI: 0.003 to 0.041 mmol/L) higher in the magnesium bicarbonate group compared to the placebo group. When data from all time points were analysed using a repeated measures analysis of variance there was a greater statistically significant difference between groups for serum magnesium (p=0.015).

Additional statistics for analyses of change showed that the mean level of magnesium across Days 14, 42 and 84 was higher among clinical trial patients receiving magnesium bicarbonate than trial patients in the placebo group after adjusting for baseline levels (0.0187 mmol/L; p=0.0070).

Conclusion and Discussion: Specific aqueous alkaline magnesium bicarbonate solutions are a safe source of bioavailable magnesium. The consumption of specific aqueous alkaline magnesium bicarbonate solutions increases serum magnesium concentrations.

This is the first successful experiment anywhere in the world to correlate, with statistical significance, dietary magnesium intake to tissue levels of magnesium in the body when the magnesium was consumed at or near the recommended daily allowance (RDA) for magnesium.

Numerous previous attempts to correlate dietary magnesium intake to tissue levels of magnesium have failed. There have been numerous international prospective studies, involving hundreds of thousands of people, that have examined the role of nutritional magnesium deficiency in a range of metabolic and other diseases in humans (see J. Clin. Epidemiol., Vol. 48 (7), 927-940, 1995; and see American Heart Journal, Vol. 136 (3), 480-490, September, 1998; and see Arch. Intern. Med., Vol. 159, 2151-2159, Oct. 11, 1999; and see Diabetes Care, Vol. 27 (1), 59-65, January, 2004; and see Diabetes Care, Vol. 29 (10), 2238-2243, October, 2006). It was found in these studies that dietary levels of magnesium did not correlate to serum levels of magnesium (see J. Clin. Epidemiol., Vol. 48 (7), 927-940, 1995; and see American Heart Journal, Vol. 136 (3), 480-490, September, 1998; and see Arch. Intern. Med., Vol. 159, 2151-2159, Oct. 11, 1999). The lack of correlation between dietary magnesium intake and tissue levels of magnesium is considered to be a result of renal influence on the complexities of magnesium homeostasis when magnesium intake is within the normal dietary range. It is known that a typical Western diet is characterised by a chronic, sub-clinical (mild) deficit in magnesium intake with about 70 per cent of people not meeting the recommended daily allowance (RDA) for magnesium (see USDA. 1999. Continuing Survey of Food Intakes by Individuals, 1994-1996. Food Surveys Research Group, Agricultural Research Service. http://www.barc.usda.gov/bhnrc/foodsurvey/pdf/Supp.pdf).

When magnesium is consumed in high pharmacological doses exceeding 3 g to 5 g per Day (8 to 12 times the RDA), elevated serum levels of magnesium have been recorded. One study was able to increase serum magnesium in normal male volunteers with lower pharmacological levels of about 4 to 5 times the RDA (see J. Am. Coll. Nutr., Vol. 13, 447-454, 1994). Pharmacological doses of magnesium produce side-effects in humans and can be consumed only for short periods of time with any safety. A serum level of magnesium exceeding 2 mM is considered toxic (see Interpretation of Diagnostic Tests, page 66, Seventh Edition, 2000, Lippincott Williams & Wilkins, Philadelphia).

### Creatinine

There was a statistically significant difference in change from baseline to Day 84 in the mean level of serum creatinine (p=0.0298) between the two treatment groups. The mean increase in serum creatinine was 3.00 μmol/L (95% CI: 0.30 to 5.69 μmol/L) higher in the magnesium bicarbonate group compared to the placebo group. When data from all time points were analysed using a repeated measures analysis of variance there was still a statistically significant difference between groups for serum creatinine (p=0.030).

Additional statistics for analyses of change showed that there was a statistically significant difference between treatment groups in the mean level of serum creatinine across Days 14, 42 and 84 with the mean level being 2.6455 μmol/L higher among clinical trial patients receiving magnesium bicarbonate than trial patients in the placebo group after adjusting for baseline levels (p=0.0139).

Conclusion and Discussion: The consumption of specific aqueous alkaline magnesium bicarbonate solutions increases serum creatinine concentrations. In the body, both creatine and phosphocreatine (creatine phosphate) may be dehydrated and cyclized to form creatinine in non-enzymatic reactions in cells. Creatinine is the breakdown product of creatine and phosphocreatine and can be detected in serum or plasma. Approximately 2 per cent of the body pool of creatine and phosphocreatine spontaneously dehydrates and cyclizes to creatinine each day. In the absence of muscle, kidney, liver and tissue pathology an increase in serum creatinine is a reflection of an increase in intracellular creatine or an increase in intracellular phosphocreatine (as an energy store), particularly in skeletal muscle and heart muscle. The brain also contains phosphocreatine as an energy store.

Additional statistics for analyses of change showed that the correlation between the change in serum magnesium and the change in serum creatinine was consistent and positive and statistically significant over Days 14 to 84. r=0.28, r=0.29, r=0.23. p<0.05. Those patients in the clinical trial who had the largest increase in concentrations of serum magnesium had the largest increase in concentrations of serum creatinine.

It is known that intracellular cytosolic magnesium concentrations increase with increasing extracellular magnesium concentrations in a dose-dependent manner in vitro within the physiological range of magnesium (see J. Cell. Physiol., Vol. 197 (3), 326-335, Aug. 22, 2003). The consumption of specific aqueous alkaline magnesium bicarbonate solutions increases the concentration of intracellular magnesium which increases the concentration of intracellular creatine or phosphocreatine in a dose-dependent manner.

Phosphocreatine is known to be the immediate source of the energy molecule adenosine triphosphate (ATP) for many biochemical reactions in body cells including plasma membrane ion ATPases such as the Na⁺—K⁺-ATPase (the cell plasma membrane sodium-potassium pump). The plasma membrane sodium-potassium pump is responsible primarily for the establishment and maintenance of concentration gradients across the cell plasma membrane. Depending on cell type, between 30 and 70 per cent of all energy (ATP) consumption in cells is utilised to maintain appropriate sodium and potassium concentration gradients.

### Parathyroid Hormone

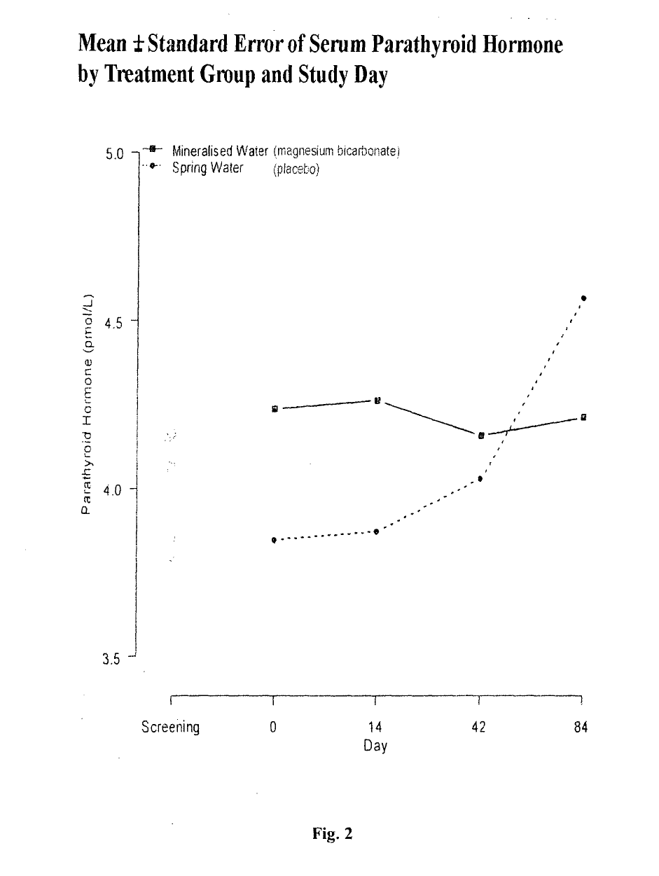

In the placebo group the mean level of parathyroid hormone increased from 3.85 pmol/L at Day 0 to 4.60 pmol/L at Day 84. In the magnesium bicarbonate group the mean level of parathyroid hormone remained relatively constant across the four time points with the mean being 4.24 pmol/L at Day 0 and 4.21 pmol/L at Day 84. There was a 0.74 pmol/L (95% Confidence Interval (CI): −0.03 to 1.52 pmol; p=0.059) difference in the change from Day 0 to Day 84 between the two groups. This is shown graphically in FIG. 2.

Additional statistics for analyses of change showed that there was a statistically significant difference in the trend in the mean level of parathyroid hormone between treatment groups between Day 0 and Day 84 (−0.0096; p=0.0363). The mean level of parathyroid hormone increased significantly among patients who received the placebo. There was no change in the mean level of parathyroid hormone in the patients who received magnesium bicarbonate.

Conclusion and Discussion: The consumption of specific aqueous alkaline magnesium bicarbonate solutions provides a source of bioavailable magnesium to stabilise serum parathyroid hormone concentrations. It is known that decreased extracellular calcium levels lead to an increase in the secretion of parathyroid hormone. Increased parathyroid hormone concentrations stimulate the release of calcium and phosphate from bone and activate retention of calcium by the kidney. In bone, increased parathyroid hormone concentrations maintain blood calcium concentrations by activating new bone remodeling units, though as a result of the inherent inefficiency of this process increased bone remodeling leads to accelerated bone loss and osteoporosis (see Basic & Clinical Endocrinology, pgs 335-338, Seventh Edition, 2004, McGraw-Hill, New York).

Parathyroid hormone is the only proven anabolic therapy for bone. However, the anabolic effect of parathyroid hormone is dependent upon either intermittent natural pulses or intermittent medical administration. When parathyroid hormone is continuously elevated, even for a few hours, it initiates processes leading to the resorption of bone which overrides any anabolic effects in relation to bone formation (see Ann. N Y Acad. Science, Vol. 1068, 458-470, April, 2006). When parathyroid hormone is administered at a frequency that permits complete clearance between doses, parathyroid hormone is anabolic for bone (see Arch. Biochem. Biophys., Vol. 473 (2), 218-224, May 15, 2008). When parathyroid hormone is present continuously at high levels, parathyroid hormone is catabolic for bone. Continuous elevated concentrations of parathyroid hormone are detrimental to the body.

Osteoporosis results from continuous elevations of parathyroid hormone. Several epidemiologic and clinical studies have shown strong associations between osteoporosis, arterial calcification and cardiovascular disease (see Clin. J. Am. Soc. Nephrol., Vol. 3 (3), 836-843, May, 2008). Vascular calcification and osteoporosis are common age-related processes that are prominently displayed on routine lateral lumbar spine radiographs as dense calcium mineral deposits of the aorta that lie adjacent to osteopenic vertebrae (see Calcif. Tissue Int., Vol. 68 (5), 271-276, May, 2001). It has been shown that people with the greatest magnitude of bone loss also demonstrate the most severe aortic calcification. It has been found also that calcifications of the aorta are directly related to bone fractures (see The Journal of Clinical Endocrinology & Metabolism, Vol. 89 (9), 4246-4253, 2004).

In the clinical trial, the placebo consisted of water devoid of calcium, magnesium, sodium and potassium (‘soft water’). It appears that the consumption of soft water at optimal volumes recommended by health professionals (1 to 2 litres per day) increases concentrations of parathyroid hormone in the body. Continuous increases in parathyroid hormone concentrations are detrimental.

### Inorganic Phosphate

There was a difference in change from baseline to Day 84 in urine inorganic phosphate concentrations between the magnesium bicarbonate group and the placebo group (p=0.0194). There was a greater decrease in urine inorganic phosphate concentration in the magnesium bicarbonate group than in the placebo group.

Additional statistics for analyses of change showed that the mean level of urine inorganic phosphate concentrations across Days 14, 42 and 84 was lower among patients receiving magnesium bicarbonate than patients in the placebo group after adjusting for baseline levels (−1.3836 mmol/L; p=0.0425).

Conclusion and Discussion: The consumption of specific aqueous alkaline magnesium bicarbonate solutions decreases urine inorganic phosphate concentration which is a reflection of stabilised serum parathyroid hormone concentrations. Parathyroid hormone strongly limits proximal tubular phosphate reabsorption in the kidneys which results in increased phosphate concentration in urine. Due to a significant increase in parathyroid hormone concentration, the group consuming placebo had significantly more urine inorganic phosphate concentration than the group consuming magnesium bicarbonate. Specific aqueous alkaline magnesium bicarbonate solutions stabilise parathyroid hormone concentrations which decreases urine inorganic phosphate concentration.

### Potassium

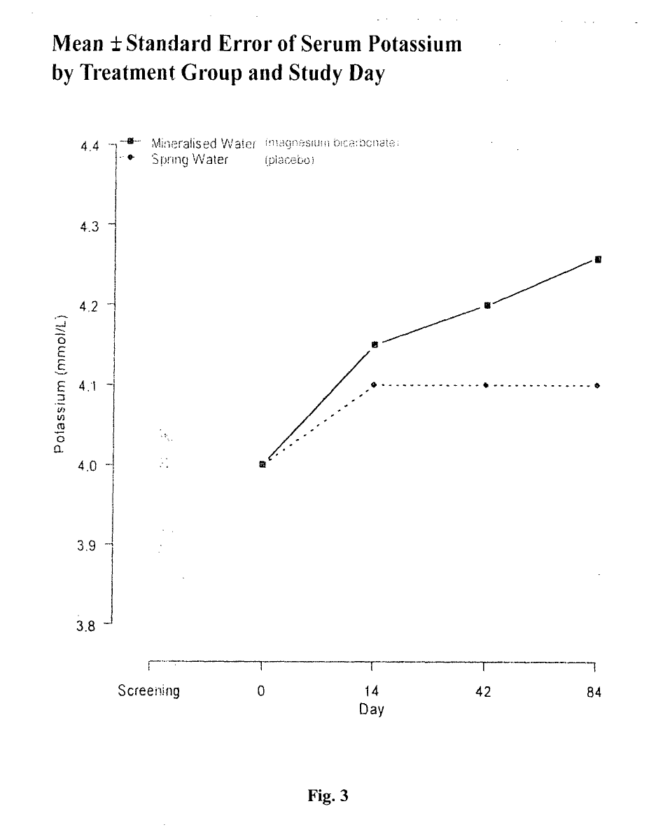

There appeared to be a trend in the difference in change from baseline to Day 84 in serum potassium concentrations between the magnesium bicarbonate group and the placebo group (p=0.1363). This is shown graphically in FIGS. 3 and 4. FIG. 3 is a result of the Statistical Analysis Plan (SAP) of the clinical trial in which the statistics highlight the difference between magnesium bicarbonate in water and water per se. The statistics incorporate adjustments for body size, diet, etc. and illustrate a difference from Day 14 to Day 84 (p=0.04). FIG. 4 shows the magnesium bicarbonate data only, placed into a linear regression (r=0.9989, p<0.002).

Additional statistics for analyses of change showed that there was a trend in the mean level of serum potassium concentrations between treatment groups between Day 0 and Day 84 (0.0018 mmol/L; p=0.0594). The mean level of serum potassium concentrations tended to increase in the magnesium bicarbonate group compared to the placebo group. An examination of change from Day 14 to Day 84 showed that the mean level of serum potassium concentrations increased in the magnesium bicarbonate group compared to the placebo group after Day 14 and was statistically significant (p=0.04).

An analysis of data showed that there was no change in the mean or median values of serum potassium concentrations from Day 14 to Day 84 in the group consuming placebo. The increase in serum potassium in the placebo group occurred in the first fourteen days of the clinical trial.

Additional statistics completed only on data from the group consuming magnesium bicarbonate showed that serum potassium concentrations increased significantly in this group throughout the trial. The Pearson correlation coefficient (r) for serum potassium concentrations from Day 0 to Day 84 in the group consuming magnesium bicarbonate was r=0.9989. A two-tailed test of significance gave p<0.002.

In the clinical trial, the mean values for the variable serum potassium were:

| | Magnesium Bicarbonate Group | Placebo Group |
|---|---|---|
| Day | K⁺ mmol/L | K⁺ mmol/L |
| 0 | 4.07 | 4.05 |
| 14 | 4.10 | 4.12 |
| 42 | 4.16 | 4.13 |
| 84 | 4.27 | 4.11 |

Conclusion and Discussion: The consumption of specific aqueous alkaline magnesium bicarbonate solutions increases serum potassium concentrations. An increase in serum potassium concentration reflects an increase in interstitial (extracellular) potassium concentration because potassium, sodium and water are continuously and freely exchanged between the plasma and the interstitial fluid across pore-lined capillary walls.

Serum concentrations of potassium increased by 5 per cent in the magnesium bicarbonate group and 1.5 per cent in the placebo group. In the group consuming specific aqueous alkaline magnesium bicarbonate solutions, body cells were in contact with interstitial fluid that contained a 5 per cent increase in potassium concentration. The group consuming magnesium bicarbonate went from low to normal (4.3 mmol/litre) serum potassium concentrations by completion of the clinical trial. [Low serum potassium is defined as less than 4.1 mmol/litre (see European Heart Journal, Vol. 28, 1334-1343, 2007).]

It is known that elevated extracellular potassium concentrations can inhibit decreases in cell volume and prevent the activation of apoptotic processes. Elevated extracellular potassium concentrations protect cells from apoptosis (see Arch. Biochem. Biophys., Vol. 462 (2), 176-188, Jun. 15, 2007). Elevated extracellular potassium protects cells from apoptosis by specifically diminishing the potassium concentration gradient across the cell plasma membrane. The consumption of specific aqueous alkaline magnesium bicarbonate solutions assists in the maintenance of cell volume and the prevention of apoptosis. This is of vital importance in the maintenance of cell and organ function and in the prevention of cell loss in organs such as the brain.

It is known that elevated extracellular potassium ion concentrations can produce relatively large changes in the intracellular to extracellular potassium ion ratio (K⁺ᵢₙ/K⁺ₒᵤₜ) and consequently in the cell resting membrane potential (negative inside). A small elevation in extracellular potassium concentration decreases the K⁺ᵢₙ/K⁺ₒᵤₜ ratio and partially depolarises the cell plasma membrane—that is, makes the resting potential less electronegative. In excitable cells such as neuromuscular tissue (nerve, muscle, heart), this increases membrane excitability because less of a depolarising stimulus is required to generate an action potential. Membrane potential is achieved normally through a complex integration of electrical charge gradients and ion concentration gradients. When inwardly rectifying potassium channels are open (the resting state) the extracellular potassium concentration is the major determinant of cell resting membrane potential (see Goodman & Gilman's The Pharmacological Basis of Therapeutics, pgs 839-843, Ninth Edition, International Edition, 1996, McGraw-Hill, New York). The consumption of specific aqueous alkaline magnesium bicarbonate solutions increases extracellular potassium concentration and decreases the magnitude of cell resting membrane potentials.

Potassium channels constitute a large and heterogeneous family of proteins that control cell plasma membrane potential. Potassium channels are recognised as potential therapeutic targets in the treatment of a range of brain and psychiatric diseases, heart diseases, muscle, bone and joint diseases, diseases associated with tumorigenesis and diseases of The Metabolic Syndrome, including diabetes (see Recent Patents CNS Drug Discov., 2(3):200-28, November 2007).

### Aquaresis and Sodium and Potassium

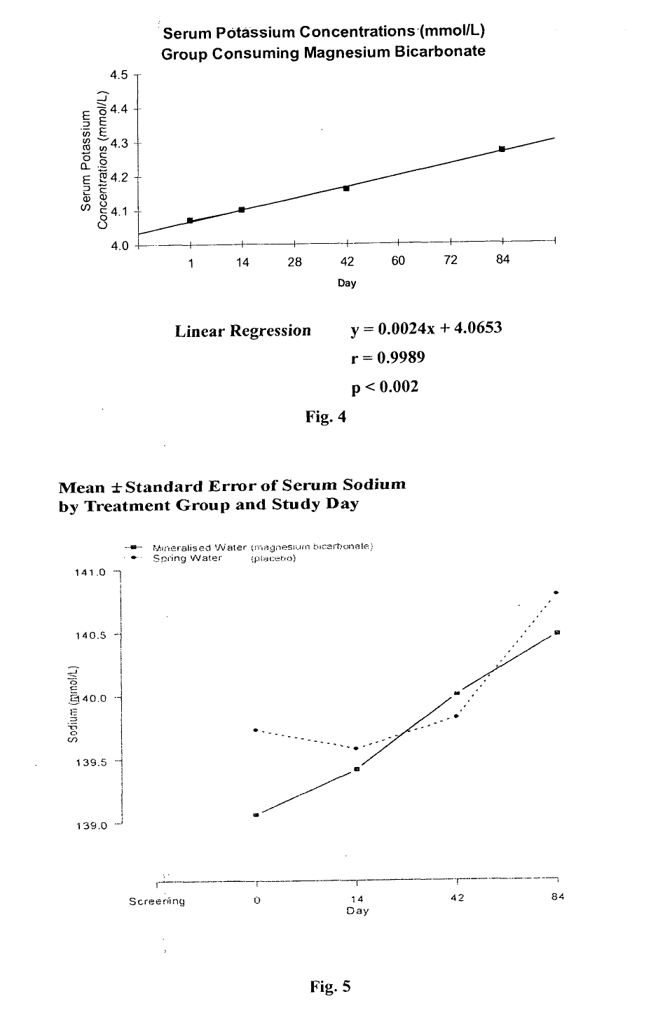

Additional analyses of change showed that there was a significant trend between Day 0 and Day 84 in the mean level of measurements in all patients in both groups for serum sodium, serum potassium and serum magnesium (see FIGS. 1, 3 and 5). There was a statistically significant increase in the mean level of serum sodium for the whole study group between Day 0 and Day 84 (0.0153 mmol/L/Day; p<0.0001). There was a statistically significant increase in the mean level of serum potassium for the whole study group between Day 0 and Day 84 (0.0015 mmol/L/Day; p=0.0028). There was a statistically significant increase in the mean level of serum magnesium for the whole study group between Day 0 and Day 84 (0.0002 mmol/L/Day; p=0.0148). It is considered that both groups in the clinical trial became optimally hydrated with the consumption of approximately 1 to 2 litres of water per day. This hydration decreased the release of antidiuretic hormone (ADH, vasopressin) from the posterior pituitary gland. A decrease in the release of vasopressin allowed essential solutes such as sodium, potassium and magnesium to be reabsorbed from the kidney tubule system without the reabsorption of water.

Conclusions and Discussion: The consumption of approximately 1 to 2 litres of water per day as placebo or as magnesium bicarbonate solution promoted aquaresis which is the electrolyte-sparing excretion of free water. With continued ingestion of 1 to 2 litres of water per day, a new steady state emerged with an aquaretic effect manifested by increased concentrations of plasma solutes.

This is the first successful experiment anywhere in the world to correlate water consumption to aquaresis. That is, this is the first successful experiment anywhere in the world to increase sodium and potassium concentrations in plasma by utilising the aquaresis that results from the consumption of optimal volumes of water. In general, in physiology and medicine, it is accepted as dogma that the consumption of water may lead to hyponatremia (low plasma sodium) and hypokalemia (low plasma potassium).

Aquaretics act generally as inhibitors of vasopressin (antidiuretic hormone, ADH), V₂ receptors. A decrease in the release of vasopressin decreases both V₁ and V₂ vasopressin receptor-effector coupling. Normally, the stimulation of V₂ receptors by vasopressin leads to the absorption of water from the collecting duct system of the kidneys.

The two components of the extracellular fluid, plasma and interstitial fluid, are separated by the walls of blood vessels including capillaries. However, water and sodium and potassium are continuously and freely exchanged between the plasma and the interstitial fluid across pore-lined capillary walls. Accordingly, plasma and interstitial fluid (extracellular fluid) are nearly identical in composition except that interstitial fluid lacks plasma proteins which cannot traverse capillary walls.

In the clinical trial, serum concentrations of sodium increased by less than 1 per cent in both the magnesium bicarbonate group and the placebo group. In contrast, serum concentrations of potassium increased by 5 per cent in the magnesium bicarbonate group and 1.5 per cent in the placebo group. In the group consuming specific aqueous alkaline magnesium bicarbonate solutions, body cells were in contact with interstitial fluid that contained a 5 per cent increase in potassium concentration. This allowed body cells to maintain hydration and cell volume and to be protected from apoptosis. Indeed, it has been demonstrated routinely in many experiments that elevated extracellular potassium concentrations protect cells from cell shrinkage and apoptosis (see Arch. Biochem. Biophys., Vol. 462 (2), 176-188, Jun. 15, 2007).

The use of water to deliver essential solutes or to increase serum concentrations of solutes, and possibly to concentrate and deliver other moieties that can be filtered and reabsorbed by the kidneys, has medical, pharmacological and nutritional implications.

The consumption of specific aqueous alkaline magnesium bicarbonate solutions modulates the overall ionic strength of the extracellular and intracellular environment and maintains cellular function. Extracellular and intracellular ionic strength controls many diverse cellular functions, particularly functions associated with electrostatic and surface charges such as enzyme function and membrane function.

### Albumin

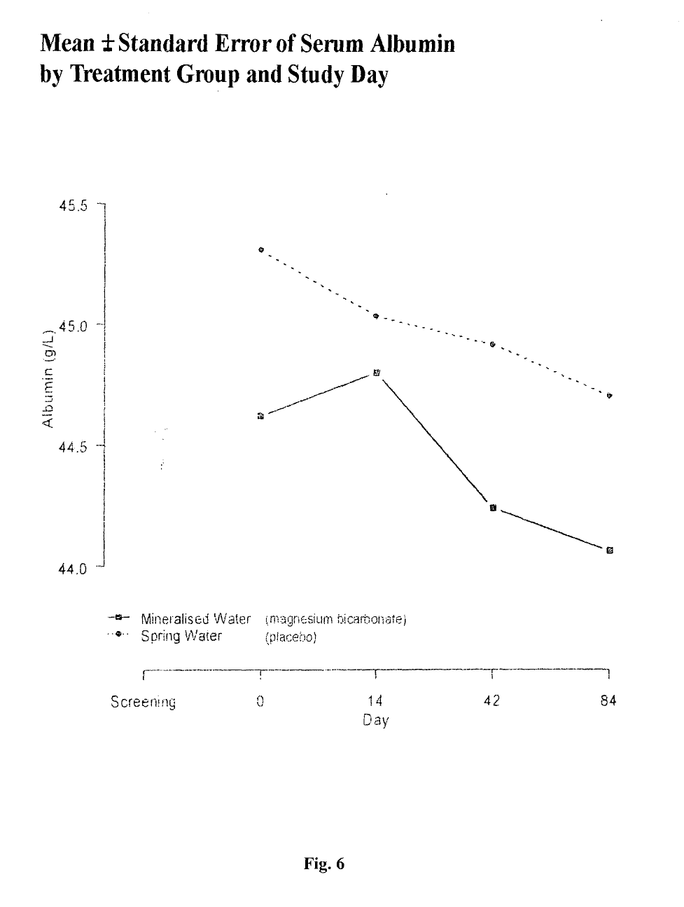

Additional statistics for analyses of change showed that the trend in the mean level of serum albumin for both the magnesium bicarbonate and the placebo group approached statistical significance at p=0.0559. Serum albumin tended to decrease in both groups. This is shown graphically in FIG. 6.

The correlation between the change in serum magnesium and the change in serum albumin was consistent and positive and statistically significant over Days 14 to 84. r=0.44, r=0.20, r=0.42. p<0.05. Those patients in the clinical trial who had the largest changes in concentrations of serum magnesium had the smallest changes in concentrations of serum albumin.

Conclusion and Discussion: In the clinical trial, both the magnesium bicarbonate group and the placebo group consumed between 1 to 2 litres of water per day. This resulted in both groups having an increase in total body water (TBW). The increase in TBW was manifested in both groups by an increased output of urine and a decrease in the concentration of solutes in the urine. An increase in TBW results in an expansion of both intracellular and extracellular compartments and a dilution of the fluids in these compartments (see Clinical Physiology of Acid-Base and Electrolyte Disorders, Rose, B. D. and Post, T. W., pgs 241-257, Fifth Edition, 2001, McGraw-Hill, New York). Extracellular fluid, including plasma, is diluted. Plasma albumin concentrations are decreased. In the clinical trial, plasma albumin concentrations were decreased in both groups by about one percent.

The decreases in serum albumin were less amongst patients in the trial with the largest increases in serum magnesium. Decreases in albumin concentrations (that is, decreases in negative charges) were less amongst patients in the trial with the largest increases in magnesium concentrations in order to preserve appropriate plasma ionic strength and electrical neutrality in the presence of magnesium cations which possess a high positive charge density due to a small ionic radius and a relatively high positive charge (2+).

### Magnesium, Parathyroid Hormone and the Calcium-Sensing Receptor

In the clinical trial, there was a statistically significant difference in change from baseline to Day 84 in the mean level of serum magnesium between the group consuming magnesium bicarbonate and the group consuming placebo (p=0.027). This is shown graphically in FIG. 1. There was a statistically significant difference in the trend in the mean level of parathyroid hormone between the two groups between Day 0 and Day 84 (−0.0096; p=0.0363). This is shown graphically in FIG. 2. The mean level of parathyroid hormone increased significantly among patients who consumed the placebo. There was no change in the mean level of parathyroid hormone in the patients who consumed magnesium bicarbonate.

Conclusion and Discussion: It has been recognised for many years that magnesium required for proper functioning of the parathyroid glands. Both very low magnesium (hypomagnesemia) and very high magnesium (hypermagnesemia) restrict the secretion of parathyroid hormone (see Basic & Clinical Endocrinology, Greenspan F. S. and Gardner D. G., pg 298, Seventh Edition, 2004, McGraw-Hill, New York).

Functioning parathyroid glands respond to the slightest decrease in concentrations of extracellular calcium. A slight decrease in extracellular calcium results in the parathyroid glands increasing the rate of secretion of parathyroid hormone (PTH) within minutes. A decrease in extracellular (plasma) calcium may be undetectable per se by standard laboratory assessments but may cause detectable increases in plasma PTH concentrations. A decrease of a fraction of a milligram per decilitre (a decrease of about 0.025 mM per litre) in plasma calcium can double PTH secretion (see Textbook of Medical Physiology, Guyton, A. C. and Hall, J. E., pgs 994-995, Ninth Edition, 1996, W.B. Saunders Company, Philadelphia).

The clinical trial demonstrated that a miniscule fall in extracellular calcium concentration, resulting from dilution of extracellular compartments (due to an increase in TBW), was sufficient to increase the plasma concentration of PTH. However, when the plasma concentration of magnesium was elevated slightly (by about 3 per cent) in the magnesium bicarbonate group the PTH response was blunted. It appears that within the normal physiological concentrations of plasma calcium and plasma magnesium, a slight elevation in plasma magnesium in vivo negates the parathyroid gland's response to a fall in extracellular calcium concentration.

This is the first successful experiment anywhere in the world to demonstrate that a slight elevation in plasma magnesium may act as an agonist to the extracellular calcium-sensing receptor in vivo.

The extracellular calcium-sensing receptor belongs to the ‘family C’ of G-protein coupled receptors. Activation of calcium-sensing receptors triggers signaling pathways that modify numerous cell functions such as proliferation, chemotaxis, apoptosis and differentiation. In the parathyroid gland, activation of the calcium-sensing receptor by elevated calcium concentrations results in suppression of PTH release. Therefore, in the parathyroid gland, stimulus-secretion coupling is manifested by an increase in intracellular calcium with suppression of hormone (PTH) secretion. This suppression is in contrast to the stimulatory effect of increases in intracellular calcium observed in most secretory processes (see Am. J. Physiol. Endocrinol. Metab., Vol. 290, E761-E770, 2006). In addition, in contrast to the calcium-sensing receptor of the parathyroid glands that inhibits the release of PTH, the calcium-sensing receptor on cancer cells (particularly breast cancer and prostate cancer cells) stimulates the secretion of parathyroid hormone related protein (PTHrP). Elevated extracellular calcium surrounding these cancer cells up-regulates PTHrP synthesis and PTHrP release via calcium-sensing receptor activation.

Magnesium has been reported as a partial agonist of the calcium-sensing receptor in vitro in contrast to calcium and barium that are full agonists. The partial agonist effect of magnesium in vitro is not detectable below a magnesium concentration of 0.5 mM and appears to require a co-existing calcium concentration of at least 0.5 to 1.5 mM. With calcium per se, there appears to be dramatic cooperativity with steadily increasing calcium concentrations as assessed by the responses to activation of the calcium-sensing receptor (see Journal of Biological Chemistry, Vol. 271 (11), 5972-5975, Mar. 15, 1996).

In the clinical trial, the agonist effect of magnesium in vivo was detectable at a plasma magnesium concentration between 0.87 mM to 0.90 mM per litre. However, a mean increase in plasma magnesium concentration of 0.028 mM in the magnesium bicarbonate group (an increase from 0.872 mM to 0.900 mM per litre) elicited the largest agonist effect on the calcium-sensing receptor of the parathyroid gland. In the clinical trial, it appeared that rising concentrations per se in plasma magnesium elicited an initial agonist effect. It could be interpreted that an increase in plasma (extracellular) magnesium concentration alters the sensitivity of the calcium-sensing receptor to changes in calcium concentration. This may occur by magnesium acting at a different site on the calcium-sensing receptor than calcium (see Journal of Biological Chemistry, Vol. 271 (11), pages 5972-5975, Mar. 15, 1996).

Because the calcium-sensing receptor is integral to the secretion or suppression of PTH from the parathyroid gland and the secretion or suppression of PTHrP in various tissues, modulation of the calcium-sensing receptor by small rises in plasma (extracellular) magnesium concentration may have implications for those pathological conditions that have a requirement for excess secretion of PTH or PTHrP. Pathological conditions that have been correlated to excess secretion of PTHrP include atherosclerosis, vascular calcification, osteoarthritis and the metastases to bone of breast and prostate and other cancers (see Am. J. Physiol. Endocrinol. Metab., Vol. 290, E761-E770, 2006; and see Endocrinology, Vol. 141 (12), 4357-4364, 2000; and see Am. J. Physiol. Endocrinol. Metab., Vol. 281, E1267-E1274, 2001).

In marked contrast to PTH which is produced only in the parathyroid glands, PTHrP is found in many tissues in both foetuses and adults, including epithelia, mesenchymal tissues, endocrine glands, and the central nervous system (see The New England Journal of Medicine, Vol. 342 (3), 177-185, Jan. 20, 2000). The relationship between the calcium-sensing receptor and the secretion of PTHrP is complex. The calcium-sensing receptor has been shown to inhibit secretion of PTHrP in normal mammary epithelial cells and participate in the regulation of calcium and bone metabolism during lactation. In contrast to normal breast cells, the calcium-sensing receptor stimulates PTHrP production by breast cancer cells. The switch by the calcium-sensing receptor from inhibition to stimulation appears mediated by intracellular cyclic AMP (see J. Biol. Chem., Vol. 283 (36), 24435-24447, Sep. 5, 2008).

### EXAMPLE 2

Medical trials were conducted involving five hundred and twenty nine (529) patients in Australia, Canada and USA. Patients in the medical trials had a range of medical conditions or diseases including diseases of The Metabolic Syndrome, particularly Type 2 diabetes; pancreatitis; muscle diseases; traumatic injury to muscles, bones and joints due to car accidents, other accidents and sports injuries; wear and tear injuries to muscles, bones and joints; bone and joint diseases such as osteoporosis and osteoarthritis; asthma; and brain and psychiatric disorders. In addition, many patients were suffering from primary tumors (cancers) or from tissue catabolism due to tumorigenesis and metastases or tissue catabolism due to chronic disease. Eight patients suffered from infection with human immunodeficiency virus (HIV) or from Acquired Immunodeficiency Syndrome (AIDS).

The medical conditions or diseases were diagnosed medically utilising protocols complying with best medical practise. In addition, the medical conditions or diseases were characterised by biochemical biomarkers that included magnesium deficiency or deficit or abnormality, creatine or creatine phosphate (phosphocreatine) deficiency or abnormality, parathyroid hormone (PTH) excess or abnormality, sodium deficiency or abnormality, potassium deficiency or abnormality, antidiuretic hormone excess or abnormality and conditions characterised by abnormal levels of parathyroid hormone related protein (PTHrP).

A large range of clinical improvements were reported across all disease conditions. Biomarkers of anabolism increased and biomarkers of catabolism decreased. Clinical improvements regressed upon cessation of the consumption of specific alkaline magnesium bicarbonate solutions.

Some patients in Australia and Canada consumed specific alkaline magnesium bicarbonate solutions for the treatment of chronic headaches including migraine headaches. Improvements were noted in the severity and frequency of headaches in all cases.

Some patients in Australia and Canada consumed specific alkaline magnesium bicarbonate solutions for the treatment of methylxanthines (particularly caffeine) toxicity. Improvements were noted in both clinical signs and biochemical profiles.

Some patients in Australia and Canada consumed specific alkaline magnesium bicarbonate solutions for the treatment of ethanol toxicity and hangover. Improvements were noted in both clinical signs and biochemical profiles.

The patients in the medical trials consumed specific alkaline magnesium bicarbonate solutions for one to two years.

The magnesium bicarbonate consumed was in aqueous alkaline solutions with pH values between pH 7.0 and pH 9.5. Magnesium cations were present in solution in concentrations ranging from 50 mg per litre to 250 mg per litre. Bicarbonate anions were present in concentrations from 200 mg per litre to 1,500 mg per litre. The magnesium bicarbonate was consumed in amounts that resulted in the consumption of between 50 mg and 300 mg magnesium per day.

The results of treatment of medical conditions or diseases are listed below:

### 1. Atherosclerosis

Atherosclerosis overwhelmingly contributes to more mortality and more serious morbidity in the Western world than any other medical or health disorder. Atherosclerosis is responsible for over half of all deaths in the Western world. The major consequences of atherosclerosis are ischemic heart disease (coronary heart disease), myocardial infarction (heart attack), cerebral infarction (stroke), aortic aneurysms, and gangrene of the extremities. It is considered also that atherosclerosis may contribute to Alzheimer's disease and other diseases of the brain related to ischemia. Atherosclerosis is a process that leads to the progressive thickening of the intimal layer of arteries with plaque formation and eventual occlusion of the arterial lumen. The term atherosclerosis is often used interchangeably with the term arteriosclerosis which means ‘hardening of the arteries’.

Carotid intima media thickness (IMT), measured non-invasively by ultrasonography, is a well-established index of atherosclerosis. Increased carotid IMT is directly associated with an increased risk for cardiovascular disease in the general population.

Eighty (80) patients of mixed ethnicity and gender who had been medically diagnosed with atherosclerosis consumed specific alkaline magnesium bicarbonate solutions for one to two years. All patients had been treated previously, following standard medical and health protocols, with either minimal benefit or undesirable side effects. Where patient compliance was possible, clinical assessments and biomarker assessments were completed both prior to consumption (Baseline) and at the termination of consumption of alkaline magnesium bicarbonate solutions. Assessments included the measurement of carotid intima media thickness (IMT) by ultrasonography and the measurements of serum concentrations of magnesium, potassium, parathyroid hormone, high-density lipoprotein (HDL) cholesterol, low-density lipoprotein (LDL) cholesterol, and the measurements of plasma concentrations of C-reactive protein (CRP), interleukin 6 (IL-6), and tumor necrosis factor α receptor 2 (TNF-α-R2).

Most clinical and biomarker assessments of atherosclerosis showed improvements in most patients who completed the consumption of alkaline magnesium bicarbonate solutions for at least one year.

Results are shown in Table 1.

### 2. Diabetes (Type 2 Diabetes Mellitus)

Diabetes mellitus is a chronic disorder of carbohydrate, fat and protein metabolism. The disease is characterised by impaired glucose metabolism and resultant hyperglycaemia. About three to five per cent of the World's population suffers from diabetes. About 80 to 90 per cent of people with diabetes have Type 2 diabetes, also called non-insulin dependent diabetes mellitus (NIDDM) and previously referred to as adult-onset diabetes. Type 2 diabetes is characterised specifically by normal or increased blood insulin levels, co-existing with a decreased ability of peripheral tissues to respond to insulin (tissue insulin resistance). About 80 per cent of Type 2 diabetics are obese, particularly suffering from abdominal obesity.

Forty five (45) patients of mixed ethnicity and gender who had been medically diagnosed with diabetes consumed specific alkaline magnesium bicarbonate solutions for one to two years. All patients had been treated previously, following standard medical and health protocols, with either minimal benefit or undesirable side effects. Where patient compliance was possible, clinical assessments and biomarker assessments were completed both prior to consumption (Baseline) and at the termination of consumption of alkaline magnesium bicarbonate solutions. Assessments included the measurements of serum concentrations of magnesium, potassium, parathyroid hormone, high-density lipoprotein (HDL) cholesterol, low-density lipoprotein (LDL) cholesterol and the measurements of plasma fasting glucose and glycosylated haemoglobin (HbA 1c).

Most biomarker assessments of diabetes showed improvements in most patients who completed the consumption of alkaline magnesium bicarbonate solutions for at least one year.

Results are shown in Table 2.

### 3. The Metabolic Syndrome

The pathophysiology of The Metabolic Syndrome is complex and has not yet been elucidated. People affected are generally aging, obese, sedentary and possess a degree of insulin resistance. Inflammatory biomarkers are often increased.

The diseases of atherosclerosis, ischemic heart disease, kidney disease and Type 2 diabetes mellitus often occur as consequences of The Metabolic Syndrome. Nearly 50 per cent of people over the age of 50 are affected in Western societies.

Fifty eight (58) patients of mixed ethnicity and gender who had been medically diagnosed with The Metabolic Syndrome consumed specific alkaline magnesium bicarbonate solutions for one to two years. All patients had been treated previously, following standard medical and health protocols, with either minimal benefit or undesirable side effects. Where patient compliance was possible, clinical assessments and biomarker assessments were completed both prior to consumption (Baseline) and at the termination of consumption of alkaline magnesium bicarbonate solutions. Assessments included the measurements of serum concentrations of magnesium, potassium, parathyroid hormone, high-density lipoprotein (HDL) cholesterol, low-density lipoprotein (LDL) cholesterol, triglycerides and the measurements of plasma fasting glucose and glycosylated haemoglobin (HbA 1c). In addition, inflammatory biomarkers were measured that included plasma C-reactive protein (CRP), plasma interleukin 6 (IL-6) and plasma tumor necrosis factor α receptor 2 (TNR-α-R2).

Most biomarker assessments of The Metabolic Syndrome showed improvements in most patients who completed the consumption of alkaline magnesium bicarbonate solutions for at least one year.

Results are shown in Table 3.

### 4. Osteoarthritis and Osteoporosis

Osteoarthritis is known also as degenerative joint disease. Osteoarthritis is characterised by the progressive erosion of articular cartilage. Osteoarthritis can be detected medically by X-ray demonstration of bone spurs or decreased joint space.

Osteoporosis is a disease causing a reduction in bone mass. The associated structural changes in bone predispose the bone to fractures. Osteoporosis can be detected medically by a bone density scan (densitometry scan) where a comparison is made to a healthy person and a score or scores (T-score and Z-score) are given.

Both osteoarthritis and osteoporosis have aberrations in metabolism as major components of their pathogenesis. Indeed, osteoporosis is regarded as one of the most common metabolic diseases per se and is the most common medical problem in older women—occurring in fifty per cent of women in their lifetime.

In osteoarthritis, there is aberrant metabolic activity associated with both joint cartilage and subchondral bone. There are large peripheral growths of bone and cartilage called osteophytes which are thought to represent the bone's attempt to grow a new articular surface.

Sixty (60) patients of mixed ethnicity and gender who had been medically diagnosed with either osteoarthritis or osteoporosis consumed specific alkaline magnesium bicarbonate solutions for one to two years. All patients had been treated previously, following standard medical and health protocols, with minimal benefit or undesirable side effects. Where patient compliance was possible, clinical assessments and biomarker assessments were completed both prior to consumption (Baseline) and at the termination of consumption of alkaline magnesium bicarbonate solutions. Assessments included the measurements of serum concentrations of magnesium, parathyroid hormone, 25 dihydroxyvitamin D, osteocalcin, corrected calcium and the measurements of plasma C-reactive protein and urinary OH proline and deoxypyridinoline concentrations.

Most clinical and biomarker assessments of osteoarthritis and osteoporosis showed improvements in most patients who completed the consumption of alkaline magnesium bicarbonate solutions for at least one year.

Results are shown in Table 4.

### 5. Tissue Catabolism

The body may be considered as being in a constant flux of anabolic and catabolic processes. As people age, there are progressive deficits in organ function and catabolic processes predominate. Chronic inflammation and other chronic medical conditions also contribute to catabolic processes. Medical research has identified magnesium depletion in persons with some chronic diseases. There is often a reduction in serum bicarbonate.

One hundred and ninety (190) patients of mixed ethnicity and gender who had been diagnosed with conditions leading to catabolism-and debility consumed specific alkaline magnesium bicarbonate solutions for one to two years. All patients were being treated, or had been treated previously, following standard medical and health protocols with minimal benefits or undesirable side effects. The catabolic conditions included senescence, chronic inflammatory conditions, primary cancers, cancer metastases, pancreatitis, human immunodeficiency virus (HIV) infection and Acquired Immunodeficiency Syndrome (AIDS).

Where patient compliance was possible, clinical assessments and biomarker assessments were completed both prior to consumption (Baseline) and at the termination of consumption of alkaline magnesium bicarbonate solutions. Assessments included the measurements of serum concentrations of magnesium, bicarbonate, alanine amino transferase (ALT), aspartate amino transferase (AST), alkaline phosphatase (ALP), gamma glutamyl transferase (GGT), lactate dehydrogenase (LD), urea nitrogen (BUN) and whole venous blood carbon dioxide (pCO₂).

Biomarker assessments of tissue catabolism showed improvements in most patients who completed the consumption of alkaline magnesium bicarbonate solutions for at least one year.

Results are shown in Table 5.

### 6. Traumatic Injuries to Muscles, Bones and Joints Due to Car Accidents, Other Accidents and Sports Injuries, and Wear-and-Tear Injuries to Muscles, Bones and Joints

Twenty two (22) patients of mixed ethnicity and gender who were suffering the results of traumatic or wear-and-tear injuries to muscles, bones and joints consumed specific alkaline magnesium bicarbonate solutions for one to two years. Medical research has identified increased serum alkaline phosphatase (ALP) activity in people with chronic bone and joint injuries.

Where patient compliance was possible, clinical assessments and biomarker assessments were completed both prior to consumption (Baseline) and at the termination of consumption of alkaline magnesium bicarbonate solutions. Biomarker assessments included serum concentrations of magnesium, potassium, bicarbonate, creatinine and alkaline phosphatase (ALP).

There were subjective and clinical improvements, to various degrees, in all patients. Assessments by physiotherapists, following standard protocols, confirmed improvements in all patients. Biomarker assessments showed improvements in the majority of patients. Improvements occurred in serum concentrations of magnesium (increased, p<0.05, n=16), potassium (increased, p<0.05, n=16), bicarbonate (increased, p<0.05, n=16), creatinine (increased, p<0.05, n=16) and alkaline phosphatase (decreased, p<0.05, n=16).

### 7. Asthma

Twenty five (25) patients of mixed ethnicity and gender who had been medically diagnosed with asthma consumed specific alkaline magnesium bicarbonate solutions for one to two years. Medical research has identified magnesium depletion in persons with acute and severe asthma.

Where patient compliance was possible, clinical assessments and biomarker assessments were completed both prior to consumption (Baseline) and at the termination of consumption of alkaline magnesium bicarbonate solutions. Biomarker assessments included serum concentrations of magnesium, potassium, creatinine and bicarbonate.

There were long term clinical improvements, to various degrees, in all patients. Acute asthma attacks were decreased considerably. Medication use was decreased. Improvements occurred in serum magnesium concentrations (increased, p<0.01, n=20), serum bicarbonate concentrations (increased, p<0.05, n=20) and serum creatinine concentrations (increased, p<0.05, n=20).

### 8. Chronic Headaches Including Migraine Headaches

Fifteen (15) patients of mixed ethnicity and gender who had been medically diagnosed with chronic and migraine headaches consumed specific alkaline magnesium bicarbonate solutions for one to two years. Medical research has identified magnesium depletion in persons with chronic headaches including migraine headaches.

Where patient compliance was possible, clinical assessments and biomarker assessments were completed both prior to consumption (Baseline) and at the termination of consumption of alkaline magnesium bicarbonate solutions.

There were long term subjective and clinical improvements, to various degrees, in all patients. Improvements occurred in serum magnesium concentrations (increased, p<0.01, n=12).

All patients reported that the frequency and intensity of headaches decreased. Sleep and social relationships improved in all patients.

### 9. Substance Toxicity Including Methylxanthines Toxicity and Ethanol Toxicity

Eight (8) patients of mixed ethnicity and gender who were suffering the results of substance and/or ethanol toxicity consumed specific alkaline magnesium bicarbonate solutions during the acute and recovery phases of the toxicity. Medical research has identified that ethanol toxicity is associated with metabolic acidosis and a rapid loss of magnesium from the blood and brain. Later, the severity of the ‘hangover’ resulting from ethanol intoxication is correlated to increased concentrations of antidiuretic hormone.

Where patient compliance was possible, clinical assessments and biomarker assessments were completed both prior to consumption (Baseline) and at the termination of consumption of alkaline magnesium bicarbonate solutions. Consumption of alkaline magnesium bicarbonate occurred for at least three (3) months after initial and later toxic episode/episodes. Biomarker assessments included serum concentrations of magnesium and bicarbonate and plasma concentrations of antidiuretic hormone (ADH, vasopressin).

There were clinical improvements, to various degrees, in all patients. Biomarker assessments showed improvements in the majority of patients. Improvements occurred in serum concentrations of magnesium (increased, p<0.05, n=8) and bicarbonate (increased, p<0.05, n=8). There were general decreases in the plasma concentrations of antidiuretic hormone (ADH, vasopressin). Though not statistically significant (p<0.1, n=8), there was a general trend in ADH decrease from 8 pg/ml plasma to 2 pg/ml plasma. Three (3) patients had large decreases in ADH from 20 pg/ml to 3 pg/ml plasma.

There were diminished side-effects to alcohol intoxication (diminished ‘hangover’, headache, nausea) in all patients.

Ten (10) patients of mixed ethnicity and gender who were suffering the results of methylxanthine (caffeine) toxicity consumed specific alkaline magnesium bicarbonate solutions during the acute and recovery phases of the toxicity. Consumption of alkaline magnesium bicarbonate occurred for at least three (3) months after the toxic episodes.

In addition to consuming alkaline magnesium bicarbonate solutions, all patients decreased their consumption of caffeine. There were clinical improvements in all patients. Headaches, tinnitus and dizziness disappeared within several weeks.

Improvements occurred in serum concentrations of magnesium (increased, p<0.05, n=10) and bicarbonate (increased, p<0.05, n=10).

### 10. Miscellaneous Conditions Including Mood Disorders

Sixteen (16) patients of mixed ethnicity and gender who were suffering from miscellaneous psychological and psychiatric conditions (mood disorders) consumed specific alkaline magnesium bicarbonate solutions for one to two years.

Where patient compliance was possible, clinical assessments and biomarker assessments were completed both prior to consumption (Baseline) and at the termination of consumption of alkaline magnesium bicarbonate solutions.

Results were mixed in relation to the degree of improvement. However, no patient in the trial regressed. In patients with severe mood disorders, there were improvements in disposition and demeanour and improvements in compliance in relation to treatment medications.

There were improvements in sleep patterns in all patients. There were improvements in social relationships in all patients.

Improvements occurred in serum concentrations of magnesium (increased, p<0.05, n=12) and bicarbonate (increased, p<0.05, n=12). Interestingly, there were significant decreases in plasma concentrations of antidiuretic hormone (ADH, vasopressin). Decreases occurred in plasma ADH from a mean of 6 pg/ml plasma to a mean of 2.5 pg/ml plasma (p<0.05, n=12). These decreases in ADH occurred independent of the time lapse between magnesium bicarbonate consumption and the testing of plasma. That is, independent of hydration.

### 11. Aberrations in Acid Base Balance

Eighty-five (85) patients from the above described medical trials, who were considered to have aberrations in acid base balance as determined by urinary pH values, were tested for change in urinary pH values over time. Patients consumed specific alkaline magnesium bicarbonate solutions for one to two years.

There were no changes in urinary pH values in the first six (6) weeks of the consumption of magnesium bicarbonate. After six (6) weeks, urinary pH values increased and the increases were statistically significant by twelve (12) weeks (increase in urinary pH value, p<0.01, n=85). After one year, the increase in urinary pH was highly significant (increase in pH value, p<0.0001, n=68).

The consumption of specific alkaline magnesium bicarbonate solutions restores acid base balance in patients suffering acid base aberrations.

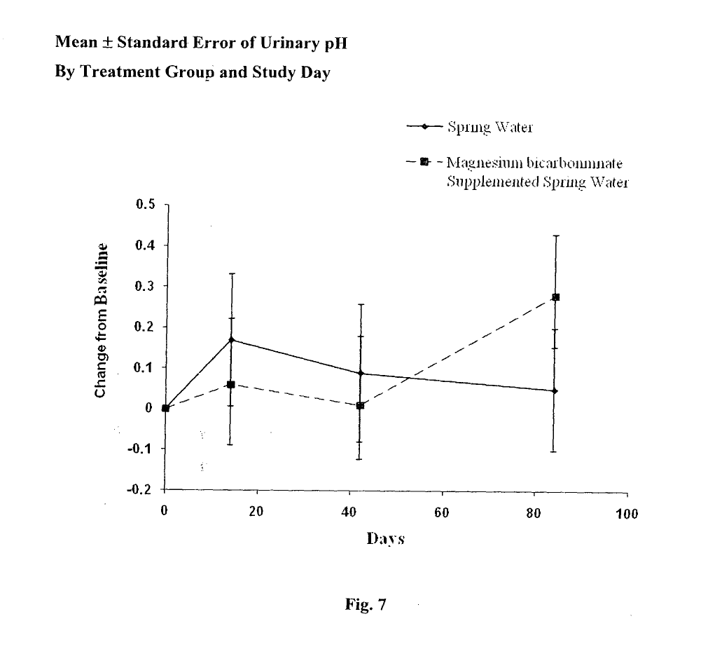

As a general example of an increase in urinary pH value over time with the consumption of specific alkaline magnesium bicarbonate solutions, see FIG. 7. FIG. 7 is derived from results of the clinical trial described in Example 1.

The difference in change from Day 0 to Day 84 for urinary pH in the magnesium bicarbonate supplemented water group was significantly different compared to the change in the non supplemented spring water control group (p=0.0182). The difference in change occurred following 42 days of consuming the magnesium bicarbonate supplemented water.

In the clinical trial, the alkaline load from the bicarbonate component (650 mg/L) of the magnesium bicarbonate supplemented water was sufficient to change urinary pH values, statistically significant by Day 84, as an indicator of acid base balance.

TABLE 1
Clinical and Biomarker Assessments for Atherosclerosis

| | | | Number of | Mean ± SD | *Student's t- |
|---|---|---|---|---|---|
| | | | observations/ | measurement | test |
| | Number of | | patients at | at | significance |
| | observations/ | Mean ± SD | termination | termination | between |
| | patients at | Baseline | of trial (1-2 | of trial (1-2 | Means (two- |
| | Baseline | measurement | years) | years) | tailed) |
| Ultrasonography right carotid intima media thickness (IMT) | 15 | 0.98 ± 0.49 | 12 | 0.84 ± 0.41 | p < 0.01 |
| (mm) | | | | | |
| Ultrasonography left carotid intima media thickness (IMT) | 15 | 0.94 ± 0.53 | 12 | 0.81 ± 0.47 | p < 0.05 |
| (mm) | | | | | |
| Serum magnesium (mmol/L) | 80 | 0.81 ± 0.19 | 58 | 0.88 ± 0.17 | p < 0.001 |
| Serum potassium (mmol/L) | 80 | 3.98 ± 0.41 | 58 | 4.37 ± 0.52 | p < 0.001 |
| Serum parathyroid hormone (PTH) (pmol/L) | 45 | 4.85 ± 3.19 | 28 | 4.26 ± 3.59 | p < 0.001 |
| Serum high-density lipoprotein (HDL) cholesterol | 35 | 1.71 ± 1.48 | 32 | 1.78 ± 1.35 | no signficance |
| (mmol/L) | | | | | |
| Serum low-density lipoprotein (LDL) cholesterol (mmol/L) | 35 | 4.86 ± 1.21 | 32 | 4.65 ± 1.98 | p < 0.01 |
| Plasma C-reactive protein (CRP) (mg/L) | 15 | 2.01 ± 1.46 | 12 | 1.65 ± 1.36 | p < 0.05 |
| Plasma interleukin 6 (IL-6) (pg/mL) | 15 | 3.21 ± 3.11 | 12 | 2.51 ± 2.78 | p < 0.05 |
| Plasma tumor necrosis factor α receptor 2 (TNF-α-R2) | 15 | 2805 ± 1005 | 12 | 2302 ± 488 | p < 0.001 |
| (pg/mL) | | | | | |

*Independent two-sample t-test

TABLE 2
Biomarker Assessments for Diabetes (Type 2 diabetes mellitus)

| | | | Number of | Mean ± SD | *Student's |
|---|---|---|---|---|---|
| | | | observations/ | measurement | t-test |
| | | | patients at | at | significance |
| | Number of | Mean ± SD | termination | termination | between |
| | observations/patients | Baseline | of trial (1-2 | of trial (1-2 | Means |
| | at Baseline | measurement | years) | years) | (two-tailed) |
| Serum magnesium (mmol/L) | 45 | 0.78 ± 0.21 | 32 | 0.83 ± 0.14 | p < 0.001 |
| Serum potassium (mmol/L) | 45 | 4.16 ± 0.38 | 32 | 4.13 ± 0.41 | no |
| | | | | | significance |
| Serum parathyroid hormone (PTH) (pmol/L) | 28 | 3.86 ± 1.10 | 18 | 3.78 ± 0.95 | no |
| | | | | | significance |
| Serum high-density lipoprotein (HDL) cholesterol | 32 | 1.37 ± 0.60 | 22 | 1.46 ± 0.57 | p < 0.01 |
| (mmol/L) | | | | | |
| Serum low-density lipoprotein (LDL) cholesterol | 32 | 4.13 ± 1.30 | 22 | 3.72 ± 1.80 | p < 0.001 |
| (mmol/L) | | | | | |
| Plasma fasting glucose (mmol/L) - patients not using | 30 | 7.4 ± 1.51 | 26 | 6.8 ± 0.82 | p < 0.001 |
| insulin | | | | | |
| Plasma fasting glucose (mmol/L) - patients using insulin | 15 | 5.7 ± 1.53 | 12 | 5.3 ± 0.91 | p < 0.01 |
| Glycosylated haemoglobin (HbA 1c) (%) | 25 | 7.2 ± 0.71 | 19 | 6.1 ± 0.58 | p < 0.001 |

| | | |
|---|---|---|
| Decrease in daily insulin doses where relevant | Baseline insulin doses | Decrease from Baseline in |
| | in 15 patients | insulin doses |
| | | in all 15 patients |

*Independent two-sample t-test

TABLE 3
Biomarker Assessments for The Metabolic Syndrome

| | | | Number of | | *Student's |
|---|---|---|---|---|---|
| | Number of | | observations/ | Mean ± SD | t-test |
| | observations/ | Mean ± SD | patients at | measurement at | significance |
| | patients at | Baseline | termination of | termination of | between Means |
| | Baseline | measurement | trial (1-2 years) | trial (1-2 years) | (two-tailed) |
| Obese | ALL | | ALL | | |
| Serum magnesium (mmol/L) | 58 | 0.79 ± 0.37 | 41 | 0.82 ± 0.58 | p < 0.05 |
| Serum potassium (mmol/L) | 58 | 4.06 ± 0.29 | 41 | 4.28 ± 0.81 | p < 0.001 |
| Serum parathyroid hormone (PTH) (pmol/L) | 19 | 4.74 ± 1.20 | 17 | 4.51 ± 1.11 | p < 0.05 |
| Serum high-density lipoprotein (HDL) cholesterol | 25 | 0.97 ± 0.17 | 19 | 1.01 ± 0.21 | p < 0.01 |
| (mmol/L) | | | | | |
| Serum low-density lipoprotein (LDL) cholesterol | 25 | 4.82 ± 1.33 | 19 | 4.62 ± 1.17 | p < 0.05 |
| (mmol/L) | | | | | |
| Serum triglycerides (mmol/L) | 25 | 1.88 ± 0.72 | 19 | 1.42 ± 0.61 | p < 0.001 |
| Plasma fasting glucose (mmol/L) | 37 | 5.91 ± 2.30 | 22 | 5.07 ± 1.92 | p < 0.001 |
| Glycosylated haemoglobin (HbA 1c) | 37 | 6.51 ± 0.92 | 22 | 6.15 ± 0.87 | p < 0.001 |
| Plasma C-reactive protein (CRP) (mg/L) | 29 | 2.12 ± 1.61 | 21 | 1.82 ± 1.01 | p < 0.001 |
| Plasma interleukin 6 (IL-6) (pg/mL) | 29 | 3.41 ± 2.90 | 21 | 2.97 ± 1.41 | p < 0.01 |
| Plasma tumor necrosis factor α receptor 2 (TNF- | 29 | 2512 ± 1152 | 21 | 2413 ± 507 | no significance |
| α-R2) (pg/mL) | | | | | |

*Independent two-sample t-test

TABLE 4
Clinical and Biomarker Assessments for Osteoarthritis and Osteoporosis

| | | | | | *Student's |
|---|---|---|---|---|---|
| | | | Number of | Mean ± SD | t-test |
| | | | observations | measurement | significance |
| | Number of | Mean ± SD | at termination | at termination | between |
| | observations | Baseline | of trial (1-2 | of trial (1-2 | Means (two- |
| | at Baseline | measurement | years) | years) | tailed) |
| Serum magnesium (mmol/L) | 58 | 0.79 ± 0.11 | 42 | 0.83 ± 0.09 | p < 0.001 |
| Serum parathyroid hormone (PTH) (pmol/L) | 58 | 5.05 ± 3.03 | 42 | 3.36 ± 2.91 | p < 0.001 |
| Plasma C-reactive protein (CRP) (mg/L) | 58 | 2.11 ± 1.21 | 42 | 1.91 ± 1.32 | p < 0.001 |
| Serum 25 Dihydroxyvitamin D (pmol/L) | 58 | 110.0 ± 43.14 | 42 | 123.0 ± 32.75 | p < 0.001 |
| Serum osteocalcin (μg/L) | 58 | 19.74 ± 3.30 | 42 | 13.01 ± 2.99 | p < 0.001 |
| Serum corrected calcium (mmol/L) | 58 | 2.29 ± 0.19 | 42 | 2.30 ± 0.16 | no |
| | | | | | significance |
| Urinary OH Proline concentration (μmol/L) | 47 | 138.0 ± 68.97 | 34 | 81.8 ± 46.18 | p < 0.001 |
| Urinary deoxypyridinoline (DPD) (nmol/mmol creatinine) | 47 | 5.74 ± 1.9 | 34 | 4.98 ± 1.68 | p < 0.001 |
| Standard clinical assessments | 58 | ** | 42 | ** | |

*Independent two-sample t-test
**stabilised or improved in all 42 patients
In-patients with osteoarthritis, there were decreased numbers and sizes of bone spurs detected.
In patients with osteoporosis, there were improvements or stabilisations in T-score and Z-score assessments.

TABLE 5
Biomarker Assessments for Tissue Catabolism

| | | | | | *Student's |
|---|---|---|---|---|---|
| | | | Number of | Mean ± SD | t-test |
| | | | observations | measurement | significance |
| | Number of | Mean ± SD | at termination | at termination | between |
| | observations | Baseline | of trial (1-2 | of trial (1-2 | Means (two- |
| | at Baseline | measurement | years) | years) | tailed) |
| Serum magnesium (mmol/L) | 190 | 0.77 ± 0.08 | 95 | 0.81 ± 0.05 | p < 0.001 |
| Serum bicarbonate (mmol/L) | 190 | 23.60 ± 3.81 | 95 | 27.72 ± 4.12 | p < 0.001 |
| Whole venous blood carbon dioxide (pCO₂) (mmHg) | 63 | 34.21 ± 5.61 | 37 | 39.81 ± 3.27 | p < 0.001 |
| Serum alanine amino tranferase (ALT) (U/L) | 190 | 33.61 ± 17.12 | 95 | 21.89 ± 11.68 | p < 0.001 |
| Serum aspartate amino transferase (AST) (U/L) | 190 | 38.57 ± 19.27 | 95 | 23.45 ± 17.61 | p < 0.001 |
| Serum alkaline phosphatase (ALP) (U/L) | 190 | 93.72 ± 37.82 | 95 | 75.56 ± 32.68 | p < 0.001 |
| Serum gamma glutamyl transferase (GGT) (U/L) | 190 | 48.73 ± 21.36 | 95 | 32.71 ± 17.25 | p < 0.001 |
| Serum lactate dehydrogenase (LD) (U/L) | 190 | 515.70 ± 237.71 | 95 | 375.60 ± 203.91 | p < 0.001 |
| Serum urea nitrogen (BUN) (mg/dL) | 190 | 30.91 ± 14.36 | 95 | 21.60 ± 9.71 | p < 0.001 |

*Independent two-sample t-test

1. A method of prevention or treatment of a condition, said method comprising administering to a patient in need thereof a therapeutic quantity of a neutral to mildly alkaline solution of a magnesium salt, said solution comprising bicarbonate ions and said condition being one that is improved by increasing bioavailable magnesium intake.

2. The method of claim 1, said condition being selected from the group consisting of magnesium deficiency or deficit or abnormality, creatine or creatine phosphate (phosphocreatine) deficiency or abnormality, parathyroid hormone (PTH) excess or abnormality, sodium deficiency or abnormality, potassium deficiency or abnormality, antidiuretic hormone excess or abnormality and conditions characterised by abnormal levels of parathyroid hormone related protein (PTHrP).

3. The method of claim 2 wherein the condition is selected from the group consisting of tissue magnesium deficiency or deficit or abnormality, tissue creatine or creatine phosphate (phosphocreatine) deficiency or abnormality, tissue parathyroid hormone excess or abnormality, tissue sodium deficiency or abnormality, tissue potassium deficiency or abnormality and tissue antidiuretic hormone excess or abnormality.

4. The method of claim 1 wherein the condition is selected from the group consisting of one that is improved by increasing body anabolism or body cell anabolism, one that is improved by decreasing body catabolism or body cell catabolism, one that is improved by both increasing body anabolism or body cell anabolism and decreasing body anabolism or body cell catabolism, ethanol toxicity, methyl xanthine toxicity, migraine, sleeplessness, psychiatric conditions requiring a calmative, pancreatitis, skin conditions, traumatic injury to a muscle, bone and/or joint, chronic headaches, a mood disorder, an aberration in acid base balance and arterial intima media thickness (IMT).

5. (canceled)

6. (canceled)

7. The method of claim 4 wherein the condition is carotid arterial intima media thickness (cIMT).

8. The method of claim 1 wherein the pH of the solution is between about 7.0 and about 9.5.

9. (canceled)

10. The method of claim 1 wherein the magnesium concentration is between about 50 and about 250 mg/L magnesium ions.

11. (canceled)

12. The method of claim 1 wherein the solution comprises about 200 to about 1500 mg/L bicarbonate ions.

13. The method of claim 1 wherein the method additionally comprises reducing or ceasing activity which causes said condition.

14. The method of claim 1 wherein said administering comprises orally administering.

15. The method of claim 1 wherein the administering is at the rate of about 1 to about 2 litres per day.

16. The method of claim 1 wherein the administering is in sufficient quantity to provide between about 50 and about 300 mg magnesium per day to said patient.

17. The method of claim 1 wherein said administration is continued for sufficient time for the condition to be alleviated or prevented.

18. The method of claim 1 wherein the patient is suffering a second condition, said second condition being one for which an increase in parathyroid hormone is adverse, and wherein said method does not result in a significant increase in parathyroid hormone.

19. The method of claim 18 wherein the second condition is selected from the group consisting of high blood pressure, ischemic heart disease, osteoporosis, atherosclerosis, The Metabolic Syndrome, Type 2 diabetes, asthma, osteoarthritis, wear and tear injuries to muscles, bones and joints.

20. The method of claim 19 wherein the condition is an inflammatory disease or a degenerative disease or myocardial infarction or stroke or virus-induced lymphocyte depletion such as human immunodeficiency virus (HIV) infection and Acquired Immunodeficiency Syndrome (AIDS).

21. The method of claim 20 wherein the condition is senescence or arthritis.

22.-28. (canceled)

29. A method for modulating the response of the parathyroid gland of a patient to a change in extracellular calcium concentration, said method comprising administering to said patient a therapeutic quantity of a neutral to mildly alkaline solution of a magnesium salt, said solution comprising bicarbonate ions.

30. The method of claim 29 wherein said modulating comprises reducing the response.

31. (canceled)

32. A method of prevention or treatment of a condition, said method comprising administering to a patient in need thereof a therapeutic quantity of a neutral to mildly alkaline solution of a magnesium salt, said solution comprising bicarbonate ions, said condition being selected from the group consisting of atherosclerosis, vascular calcification, osteoarthritis and cancer.

33. The method of claim 32 wherein the cancer is breast cancer or prostate cancer.

34. A method for preventing or inhibiting metastasis to bone of a cancer, said method comprising administering to a patient in need thereof a therapeutic quantity of a neutral to mildly alkaline solution of a magnesium salt, said solution comprising bicarbonate ions.

35. The method of claim 34 wherein the cancer is breast cancer or prostate cancer.

36.-45. (canceled)

46. A method of improving acid/base balance and/or arterial intima media thickness (IMT), said method comprising administering to a patient in need thereof a therapeutic quantity of a neutral to mildly alkaline solution of a magnesium salt, said solution comprising bicarbonate ions.
